(19) United States
(12) Patent Application Publication
(10) Pub. No.: US 2021/0008120 A1
(43) Pub. Date: Jan. 14, 2021

(54) Cell products with improved stability and uses thereof

(71) Applicant: OXACELL AG, Potsdam (DE)

(72) Inventor: Stephan BORN, Berlin (DE)

(73) Assignee: OXACELL AG, Potsdam (DE)

(21) Appl. No.: 16/980,637

(22) PCT Filed: Feb. 13, 2019
(86) PCT No.: PCT/EP2019/053585

§ 371 Date: Sep. 14, 2020

(30) Foreign Application Priority Data
Mar. 13, 2018 (EP) 18161578.2

(51) Int. Cl. A61K 35/28; A61K 35/17; A61K 35/16; A61K 47/02; A61J 1/05; A61J 1/10; A61M 5/32

(52) U.S. Cl. CPC A61K 35/28; A61K 35/17; A61K 35/16; A61M 5/32; A61J 1/05; A61J 1/10; A61K 47/02

(57) ABSTRACT

The present invention relates to an improved method of storing cells for an extended period of time as well as cell products comprising formulations of cells that have an improved stability, particularly under chilled conditions. These cell products may subsequently be used for the purpose of cell culture as well as for therapeutic applications.

### TECHNICAL FIELD

The present invention relates to an improved method of storing cells for an extended period of time as well as cell products comprising formulations of cells that have an improved stability. These cell products may subsequently be used for the purpose of therapeutic applications.

### BACKGROUND

Products comprising living cells often have to be stored and shipped under conditions which may have an impact on the viability of the cells. Living cells find application in a variety of applications, for example for the cultivation of cells that are subsequently used to produce certain factors or more cells of interest.

Other applications of living cells relate to therapeutic interventions, for example the transfer of living cells for cosmetic purposes or for restorative purposes, for example, the restoration of cell populations that have been destroyed during chemotherapy.

An area of great interest in the therapeutic field is the use of stem cells, which are cells that have been obtained from different types of sources, e.g., embryonic stem cells, adult stem cells, induced pluripotent stem cells, genetically modified stem cells of any origin, et cetera, in the treatment of certain conditions, for example diseases of different nature. Such diseases or disorders may be degenerative diseases, for example, neurodegenerative diseases or degenerative diseases of the joints, and the like. Other applications of stem cells relate, for example, to the above-mentioned restoration of affected tissues subsequent to treatment with toxic substances such as chemotherapy.

Stem cells have been isolated from large number of different tissue types or organs. One type of stem cell is the so-called mesenchymal stem cell (MSC), which has also been obtained from different types of tissues, for example from adipose tissue. The last decades have seen a multitude of publications relating to the isolation and cultivation as well as storage of stem cells. A short review relating to the preservation of stem cells is found in J. Hanna and A. Hubel, Organogenesis 5: 3, 134-137; July/August/September 2009.

Mesenchymal stem cells (MSCs) from different sources are encouraging tools for regenerative medicine because of their manifold immunomodulatory and regenerative properties [1, 2]. One of these sources is adipose tissue. Adipose tissue can easily be digested by collagenase, releasing a heterogeneous cell fraction called stromal vascular fraction [3]. Besides granulocytes, monocytes, lymphocytes, endothelial cells, pericytes, erythrocytes and other cells this fraction includes a large number of mesenchymal stem cells, called adipose derived stem cells (ASCs) [4]. MSCs derived from adipose tissue share properties similar to bone marrow derived MSCs (BM-MSCs) [5] and MSCs derived from other sources [6].

Despite the abundance of available stem cells obtainable from adipose tissue, a major problem in the establishment of cell therapies with e.g. MSC-based cell products is the shipment of the cell products, which is also due to the conditions under which the cell products have to been shipped. Conventionally, cell products were shipped under ultra-deep frozen conditions, which means at temperatures below −150° C. Such shipments are technical challenging and quite expensive. Furthermore, ultra-deep frozen cell products cannot be directly administered to patients. Instead, these products have to be further processed by the customers, i.e., medical staff intending to prepare the cells for administration. The processing of the cells can be associated with a loss in the number of cells and may also negatively influence the viability of such cells. Further, the shipment of cells under chilled rather than ultra-deep frozen conditions was also discussed in the literature [7, 8]. The problem with this type of storage condition and shipment is, that the cell products show only a short shelf-life (<48 h) when they are stored in a temperature range between about 2-8° C. [7, 8]. Due to the short shelf-life only a regional shipment is possible.

The present invention relates to products and methods of preparing formulation comprising cells of interest, in particular stem cells such as MSC, which have a shelf-life of ≥7 days under chilled conditions (2-8° C.). This invention makes possible the worldwide shipping of cell-products, such as MSC-based cell products under chilled conditions. Advantageously, chilled storage and shipping conditions are quite inexpensive, technically not very challenging and easy to establish. The inventive cell products comprising the new formulations comprising cells, for example, stem cells can be administered directly to patients and surprisingly without further processing or thawing steps. In other words, the products of the present invention, as will be explained in further detail below, are suitable for direct administration to a patient in need thereof.

Other cell types that find frequent use and have a huge potential of being used in the treatment of diseases and disorders are various types of blood cells, for example T cells. Using the present invention, i.e. the products comprising the inventive formulations, it is possible to also ship and administer these types of cells and to use them, for example in the treatment of various diseases and/or disorders.

The above-mentioned review of J. Hanna and A. Hubel describes several attempts and methods to improve stability and viability of the cells. It was known for a long time that various types of cells obtained in cell culture of primary cells, stem cells and transformed cells may be stored for a prolonged period of time in formulations comprising stabilizing ingredients such as serum, plasma, sugars, serum proteins, gelatine, et cetera. For example, EP 3178318A1 discloses a composition for promoting storage stability of stem cells and describes that the addition of serum or plasma improves cold storage stability of the cells so that these cells can maintain a survival rate of over 90% for lease nine days. WO 94/00567 already discloses methods of stabilizing lymphocytes and/or stem cells obtained from a mammal by suspending the same in an aqueous medium comprising gelatin and optionally plasma.

US 2012/207715 A1 discloses methods and storage media for preserving and prolonging the viability of cultured cells. The storage medium disclosed in said document comprises fibrin microbeads with cells attached to them. The method disclosed in said document is carried out at 18° C. to about 30° C. The disadvantage of the approach is that the cells may not be administered directly, but would have to be detached from microbeads before application. Furthermore, a transport temperature of 18° C.-30° C. requires a tightly controlled temperature-monitoring network for transportation making sure that the temperature does not drop below 18° C. or exceed 30° C.

Therefore, there is still room for improvement of storage and shipping stability of living cells, in particular of cells that have to be used in therapeutic applications, and which should not comprise any foreign or potentially hazardous materials for the subject to be treated with such cells. The inventors of the present application have surprisingly found a solution to this problem as set forth below.

### FIGURES

FIG. 1—Illustration of the mode of operation of the invention: Schematic description of the mode of operation of the inventive formulation/cell product consisting of cells dissolved in a solution comprising human pooled serum (liquid phase) and ambient air (visible gas phase) filled in e.g. a syringe (A), vial (B) or other container. During the storing process at 2-8° C. and oxygen (O₂) diffuses from the visible gas phase (which may comprise additional gases such as nitrogen (N2) and/or carbon dioxide (CO₂)) in the liquid phase. The liquid phase may comprise a water based solution containing pooled human serum, sodium chloride and glucose. The living cells in the solution take up the oxygen and release CO₂. The carbon dioxide may remain in dissolved state in the solution or may diffuse into the visible gas phase.

FIG. 2—Trend of the cell viability during cell storage under chilled conditions: Cells diluted in the formulations described in the present application were stored at 2-8° C. for 0-8 days. Subsequently, the cell viability was measured. The mean values of the cell viability (gray dots/dashed line) are shown. The viability of cells after harvesting and before diluting is indicated by black dots. The black dashed line shows the threshold (limit) of the cell viability which is 70%.

FIG. 3—Adipogenic differentiation of ASCs: Differentiated adipocytes of normally cultivated ASCs (upper left picture) and derived from ASCs previously stored under chilled conditions at temperatures of 2-8° C. (lower left picture) were fixed and stained with Oil Red-O-staining and compared with undifferentiated controls (right pictures). The pictures were taken under bright field with a 20× magnitude.

FIG. 4—Chondrogenic differentiation of ASCs: Differentiated chondrocytes of normally cultivated ASCs (upper left picture) and derived from ASCs previously stored under chilled conditions at temperatures of 2-8° C. (lower left picture) were fixed and stained with Alcian-blue-staining in comparison to undifferentiated controls (right pictures). The pictures were taken under bright field with a 20× magnitude.

FIG. 5—Osteogenic differentiation of ASCs: Differentiated osteoblasts of normally cultivated ASCs (upper left picture and derived from ASCs previously stored under chilled conditions at temperatures of 2-8° C. (lower left picture) were fixed and stained with Kossa-staining in comparison to undifferentiated controls (right pictures). The pictures were taken under bright field with a 20× magnitude.

FIG. 6—ACSs stained for senescence-positive cells: ASCs were fixed and stained for Senescence-associated beta-galactosidase activity with X-gal (middle column, blue to green cells). The pictures were taken under phase contrast with a 10× magnitude. Additionally, the nuclei of all cells were stained with Hoechst 33342 (left column). These pictures were taken under UV-fluorescence light (350-385 nm) with a 10× magnitude. The rightmost column shows merged pictures.

FIG. 7—Comparison of the amount of senescence-positive cell between cultivated and stored cells: Based on the ratio between the amount of senescence-positive and total cells the percentage of senescence-positive cells for normally cultivated cells (black triangle/n=3) and cells previously stored under chilled conditions at temperatures of 2-8° C. (gray dots/n=2) was calculated. The percent amount was plotted against the passage. The passage of the chilled stored and re-seeded ASCs corresponds to passage 9 of the normal cultivated cells, which means that cells of passage 5 were dissolved in the inventive formulation and filled into containers (here syringes). In parallel, a fraction of the cells was further cultivated. Cells in the syringes were re-seeded in culture flask after 7 days storage as indicated above and left to grow until confluency was achieved. By that time, the fraction of the cells that was further cultivated was in passage number 9.

FIG. 8—Comparison between secreted amounts of different mediators from cultivated ASCs and ASCs previously stored under chilled conditions: To demonstrate the therapeutic potential of re-seeded ASCs previously stored under chilled conditions (black squares/n=2), the secreted amounts of b-NGF (nerve growth factor beta), Eotaxin, HGF (hepatocyte growth factor), IL-6 (interleukin 6), IL-8 (interleukin 8), MCP-1 (monocyte chemoattractant protein 1) and TNF-α (tumor necrosis factor alpha) were measured and compared to the secreted amounts obtained from normal cultivated ASCs (gray dots/n=16). The amounts in pg/ml were normalized to the cell number of 1×10⁶ CD90+ cells. The passage of the chilled stored and re-seeded ASCs corresponds to passage 9 of the normal cultivated cells as explained above.

FIG. 9—Comparison of the different formulations for the liquid phase: The viability of mesenchymal stem cells dissolved in the following four different formulations was tested as described in Example 3:
1. Isotonic sodium chloride solution (black dots bar),
2. Glucose at a final concentration of 0.5% (w/v) (gray lines bar) in isotonic sodium chloride solution,
3. Human pooled serum at a final concentration of 10% (w/v) (white bar), and
4. Human pooled Serum at a final concentration of 10% (w/v) and Glucose in a final concentration of 0.5% (w/v) as according to the invention (black bar).
   All four different liquid phase compositions include a visible gas phase of ambient air. The gray bar represents the viability before storage (n=3).

FIG. 10—Comparison of the different compositions for the gas phase: The viability of mesenchymal stem cells in three different cell products was tested as described in Example 3:
1. Gas phase as described as herein is visible separated from the fluid phase (black bar),
2. Packaged cell product with a formulation described in EP 3178318A1 without a gas phase visible separated from the fluid phase (white bar),
3. A gas phase visible separated from the fluid phase and composed of nitrogen only (dotted bar)
4. The dashed bar represents the viability of the MSCs before storage. (n=4).

FIG. 11—Variation of the gas phase to fluid phase ratio: The viability of mesenchymal stem cells in three different cell products was tested as described in Example 3:
1. Gas phase as described according to the invention and is visibly separated from the fluid phase (black bar),
2. The volume ratio for the gas (g) and the fluid phase (f) was 1(g):1 (f). Ambient air was taken as gas phase (dark gray bar),
3. The dashed bar represents the viability of the MSCs before storage. (n=4).

FIG. 12—Comparison of the different types of containers for aseptic filling: The viability of mesenchymal stem cells filled in a micro tube for storage was tested in comparison to that of cells filled into a syringe as described in Example 3: 1. MSC filled in syringe as described as invention (black bar) with a visible gas phase, 2. MSC filled in a 1.5 ml micro tube with a visible gas phase and stored for seven days (horizontally dashed bar), 3. The diagonally lined bar represents the viability of the MSC before the storage. (n=3).

FIG. 13—Trend of the cell concentration during cell storage under chilled conditions: Cells diluted in the formulations described in the present application were stored at 2-8° C. for 0-7 days. Subsequently, the cell concentration was measured as described in Example 4. The mean values of the cell viability (black dots) are shown. The cell concentration before filling the cells into syringes is indicated by day -1 (gray dot). The dotted line represents a linear trend line.

### DEFINITIONS

In order to facilitate the understanding of the present description, the meaning of some terms and expressions in the context of the invention will be explained below. Further definition will be included through the description as necessary. Unless defined otherwise, all technical and scientific terms used herein have the same meaning as commonly understood to one of ordinary skill in the art to which this invention belongs.

As used in this specification and in the appended claims, the singular forms of “a” and “an” also include the respective plurals unless the context clearly dictates otherwise.

In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%.

It is to be understood that the term “comprising” is not limiting. For the purposes of the present invention the term “consisting of” is considered to be a preferred embodiment of the term “comprising of”. If hereinafter a group is defined to comprise at least a certain number of embodiments, this is meant to also encompass a group which preferably consists of these embodiments only.

The terms “treat, treating, treatment” as used herein and in the claims means preventing, alleviating or ameliorating diseases or disorders. The terms “prophylaxis or prevention” as used herein and in the claims refers to the administration or use of the herein disclosed formulations, compositions or cells in order to prevent the worsening, onset or aggravation of a given disease or disorder.

As used herein, the term “cell” relates to human or animal cells, preferably human cells, comprising mesenchymal human or animal stem cells, which may be obtained from different sources, e.g., fat tissue, bone marrow, umbilical cord, liver, dental pulp, synovial fluid, retinal, skeletal muscle, periodontal ligament, urine, mammary gland, peripheral blood, Wharton's jelly, periapical cyst, amnion, amniotic fluid, human or animal embryonic stem cells, induced pluripotent stem cells, fetal stem cells, precursor cells, primary cells, cell types selected from the group comprising somatic cells, T cells, natural killer T cells, genetically modified cells, e.g., CART-cells, fibroblast type cells, pre-differentiated and differentiated cells.

As used herein, the term “stem cell” relates to the commonly accepted meaning of, e.g., human or animal embryonic stem cells, induced pluripotent stem cells, fetal stem cells, tissue-derived stem cells and designates cells which are not terminally differentiated, but may still further develop into specific differentiated cell types.

As used herein the term “mesenchymal stem cell” (“MSC”) shall be taken to mean a cell which is capable of giving rise to multiple different types of cells, originally derived from the mesenchyme. The term refers to a cell which is capable to differentiating into at least one of an osteoblast, a chondrocyte, an adipocyte or a myocyte. MSC may be isolated from any type of tissue. Generally MSC will be isolated from bone marrow, adipose tissue, umbilical cord, or peripheral blood but is not limited to. The MSCs used in the invention may in some embodiments be isolated from adipose tissue (ASCs) but not limited to. In a preferred aspect of the invention, MSC are obtained from lipoaspirates themselves obtained from adipose tissue. Herein, the abbreviations MSC or ASC are used interchangeably.

As used herein, the term “container” relates to any known vessel that is suitable for the storage of cells. The container may, for example, be an injection device or part thereof, for example a syringe, an injection pen, a tube, an micro/reaction tube, a plastic flask, a vial, a phiole, a cell culture flask, cell culture bag, et cetera. As used herein, the container generally does not comprise a cell medium for in vitro culturing of cells, but comprises a formulation that is suitable for direct administration of the cells to an individual in need thereof. The formulation does not comprise any solid or particulate matter that should not be administered to an individual, e.g. beads having cells attached thereto.

As used herein, the term “formulation” relates to any artificially composed medium, which may contain cells and which may fill a container as used herein. Optionally, the formulation comprises from known additives of cell culture media, for example serum, plasma, glucose, other sugars, vitamins, salts, acids or bases for the regulation of the pH value, water, gelatin, growth factors, reconstituted artificial media, and the like. The base for the formulations according to the present invention is often physiological saline.

As used herein, the term “medium” relates to any liquid or liquid-gas-mixture that is suitable for the storage of cells as used herein.

As used herein, the term “immunomodulatory” refers to the inhibition or reduction of one or more biological activities of the immune system which includes, but is not limited to, downregulation of immune response and inflammatory states as well as changes in cytokine profile, cytotoxic activity and antibody production.

As used herein, the term “MHC” (major histocompatibility complex) refers to a subset of genes that encodes cell-surface antigen-presenting proteins. In human, these genes are referred to as human leukocyte antigen (HLA) genes. Herein, the abbreviations MHC or HLA are used interchangeably.

As used herein, the terms, “negative” or “−” as used in respect of cell surface markers shall be taken to mean that, in a cell population, less than 20%, 10% preferably less than 9%, 8%, 7%, 6%, 5%, 4%, 3%, 2%, 1% or none of the cells express said marker. Expression of cell surface markers may be determined for example by means of flow cytometry for a specific cell surface marker using conventional methods and apparatus (for example a Merck Millipore guava® easyCyte™ 6-2L flow cytometer used with commercially available antibodies and standard protocols known in the art).

As used herein, the expression “significant expression” or its equivalent terms “positive” and “+” when used in regard to cell surface marker shall be taken to mean that, in a cell population, more than 20%, preferably more than, 30%, 40%, 50%, 60%, 70%, 80%, 90%, 95%, 98%, 99% or even all of the cells express said marker. Expression of cell surface marker may be determined for example by means of flow cytometry for a specific cell surface marker using conventional methods and apparatus (for example a Merck Millipore guava® easyCyte™ 6-2L flow used with commercially available antibodies and standard protocols known in the art) that show a signal for a specific cell surface marker in flow cytometry above the background signal using conventional methods and apparatus (for example a Merck Millipore guava® easyCyte™ 6-2L flow used with commercially available antibodies and standard protocols known in the art). The background signal is defined as the signal intensity given by a non-specific antibody of the same isotype as the specific antibody used to detect each surface marker in conventional flow cytometer analysis. For a marker to be considered positive the specific signal observed is stronger than 20%, preferably stronger than, 30%, 40%, 50%, 60%, 70%, 80%, 90%, 100%, 500%, 1000%, 5000%, 10000% or above, than the background signal intensity using conventional methods and apparatus (for example a Merck Millipore guava® easyCyte™ 6-2L flow used with commercially available antibodies and standard protocols known in the art). Furthermore, commercially available and known monoclonal antibodies against said cell-surface markers (e.g. cellular receptors and transmembrane proteins) can be used to identify relevant cells.

As used herein, the term “fibroblast” as used herein shall be taken to include fibroblast like synovial cells.

The term “T-cell” refers to cells of the immune system which are a subset of lymphocytes that express the T cell receptor (TCR).

As used herein, the terms “treat”, treatment” and “treating” when used directly in reference to patient or subject shall be taken to mean the amelioration of one or more symptoms associated with a disorder including, but not limited to an, cancer, immune diseases, patients subjected to chemotherapy, organ failure, motor neuron disease, acute and chronic organ dysfunction, short- and long-term autoimmune disorders, osteoarthritis, myocardial dysfunction, systemic lupus erythematosus, psoriatic arthritis, fibromyalgia, stroke, diseases with organ degeneration, chronic obstructive lung disease, bone defects, inflammatory arthritis, spinal cord injury, limb wounds, liver fibrosis/cirrhosis, hepatobiliary diseases, neurodegenerative ophthalmic 'disorders, diabetes mellitus, inflammatory bowel diseases, graft-versus-host disease, polyneuropathy, sepsis, acute respiratory distress syndrome, non-arteritic ischemic optic neuropathy, nephropathy, wherein said amelioration results from the administration of the immunomodulatory cells of the invention, or a pharmaceutical composition or formulation comprising same, to a subject in need of said treatment.

As used herein, the term “culture” refers to any growth of cells, organisms, multicellular entities, or tissue in a medium. The term “culturing” refers to any method of achieving such growth, and may comprise multiple steps. A “cell culture” refers to growth of cells in vitro. In such a culture, the cells proliferate, but they do not organize into tissue per se.

As used herein, the term “culture media supernatant” used herein refers to cell culture media free of cultured cells/tissue, resulting after a period of time in contact with the cultured cells such that the media has been altered to include certain paracrine and/or autocrine factors produced by the cells and secreted into the culture.

As used herein, the term “confluent culture” is a cell culture in which all the cells are in contact and thus the entire surface of the culture vessel is covered, and implies that the cells have also reached their maximum density, though confluence does not necessarily mean that division will cease or that the population will not increase in size.

As used herein, the term “culture medium” or “medium” is recognized in the art, and refers generally to any substance or preparation used for the cultivation of living cells. The term “medium”, as used in reference to a cell culture, includes the components of environment surrounding the cells. Media may be solid, liquid, gaseous or a mixture of phases and materials. Media include liquid growth media as well as liquid media that do not sustain cell growth. Media also include gelatinous media such as agar, agarose, gelatin and collagen matrices. Exemplary gaseous media include the gaseous phase that cells growing on a petri dish or other solid or semisolid support are exposed to. The term “medium” also refers to material that is intended for use in a cell culture, even if it has not yet been contacted with cells.

As used herein, the term “differentiation” refers to the formation of cells expressing markers known to be associated with cells that are more specialized and closer to becoming terminally differentiated cells incapable of further divisions and or differentiation. For example, in a chondrogenic context, differentiation can be seen in the production of chondrocytes cluster that produce and maintain the cartilaginous matrix.

As used herein, the term “expanded” as used herein when referring to cells shall be taken to have its usual meaning in the art, namely cells that have been proliferated in vitro. Method for the preparation of expanded human ASCs are known in the art, for example as described in WO2007/039150. An ASC can be expanded to provide a population of cells that retain a least one biological function of the ASC, typically the ability to adhere to a plastic surface, under standard culture conditions. The expanded population of cells retains the ability to differentiate into one or more cell type and secreting certain paracrine and/or autocrine factors but are not limit to.

As used herein, the term “including” is used herein to mean “including but not limited to”. “Including” and “including but not limited to” are used interchangeably.

As used herein, the term “Marker” refers to a biological molecule whose presence, concentration, activity, or phosphorylation state may be detected and used to identify the phenotype of a cell.

As used herein, the term “phenotype” refers to the observable characteristics of a cell, such as size, morphology, protein expression etc.

As used herein, the term “progenitor cell” refers to a cell has the capacity to create progeny that are that are more differentiated then itself.

“Proliferation” refers to an increase in cell number. “Proliferating” and “proliferation” refer to cells undergoing mitosis.

As used herein, the term “solution” includes a carrier or diluent in which the cells are solved.

As used herein, the term “solvent” refers to a substance that dissolves a solute (including a chemically distinct liquid, solid or gas), resulting in a solution. A solvent is usually a liquid but can also be a solid, a gas, or a supercritical fluid.

As used herein, the term “senescence” refers to the gradual deterioration of function characteristic of most complex lifeforms, arguably found in all biological kingdoms, that on the level of the organism increases mortality after maturation. The word senescence can refer either to cellular senescence or to senescence of the whole organism.

As used herein, the term “Re-seeding” and “reseed” refers to sow cells again in order to grow them.

As used herein, the term “Formulation” as used herein shall be taken to have its usual meaning in the art, namely putting together of components in appropriate ratios compounds, percentages, relationships or structures, according to a formula. As used herein a “formulation”, includes a mixture or a structure such as a capsule, a pill, tablet, or an emulsion or any other pharmaceutically acceptable composition.

As used herein, the term “component” refers to a constituent part; element; ingredient but is not limited to.

As used herein, the terms “Ambient gas” as well as “ambient air” used herein shall be taken to have its usual meaning in the art, namely ambient air is atmospheric air in its natural state, not contaminated by air-borne pollutants. “Ambient air” is typically 78% nitrogen and 21% oxygen. The extra 1% is made up of a combination of carbon, helium, methane, argon and hydrogen. The closer the air is to sea level, the higher the percentage of oxygen. “Ambient gas” and “ambient air” are used interchangeably.

As used herein the term “Chilled condition” refers to an ambient temperature of >0 to ≤15° C., preferably of a range of 2-8° C. but not limited thereto.

As used herein, the term “Gas phase” shall be taken to have its usual meaning in the art, namely state of matter distinguished from the solid and liquid states by relatively low density and viscosity, relatively great expansion and contraction with changes in pressure and temperature, the ability to diffuse readily, and the spontaneous tendency to become distributed uniformly throughout any container. The term “gas” or “gas-phase” or derivatives thereof refers to ambient air, mixtures comprising oxygen and nitrogen, pure oxygen gas, mixtures comprising oxygen and inert gas, for example, argon, mixtures comprising pure oxygen mixed with ambient air, and the like.

As used herein, the term “fluid phase” or “liquid phase” shall be taken to have its usual meaning in the art, namely the state of matter in which a substance exhibits a characteristic readiness to flow and little or no tendency to disperse, and is amorphous but has a fixed volume and is difficult to compress. Preferably the substance is water-based but no limited thereto. The terms “fluid phase” and “liquid phase” are used interchangeably.

As used herein the term “stem cell” refers to undifferentiated biological cells that can differentiate into specialized cells and can divide (through mitosis) to produce more stem cells. They are found in multicellular organisms. In this invention stem cell from mammals preferred but not limit to. Mammalian stem cells can be grouped in two broad types of stem cells: embryonic stem cells, which are isolated from the inner cell mass of blastocysts, and adult stem cells, which are found in various tissues. In a preferred aspect of the invention, adult stem cells derived from the mesenchyme are obtained (MSC), more preferably mesenchymal stem cells obtained from lipoaspirates (ASC) but not limit to. Herein the term “stem cells” were used as hypernym including MSC and ASC but not limited to it.

The term “about” when used in relation to a value relates to the value ±10%, in particular ±5%.

As used herein, the term “composition” is used in the inclusive, open sense, meaning as the act of combining parts or elements to form a whole. The compositions of the inventions may include, in a addition to MSC, non-cellular components. Examples of such non-cellular components include but are not limited to cell culture media, which may compromise one or more proteins, amino acids, nucleic acids, nucleotides, co-enzyme, metals, salts of metals and gases.

As used herein, the term “passage” shall be taken to have its usual meaning in the art, namely the increment number of subcultures when confluent expanded cells are divided to subcultures for further expanding.

As used herein, the term “stromal vascular fraction” (also referred to herein as “svf”) shall be taken to have its usual meaning in the art, namely the heterogeneous cell fraction resulting of a digestion of adipose tissue.

As used herein the term “store” and the term “storage” refers to non-transitory, semi-permanent or long-term, containment, holding, leaving, or placement of goods or materials, usually with the intention of retrieving them at a later time. As used herein the storage was done in a controlled environment.

As used herein, the term “allogenic” as use herein shall be taken to mean different individuals of the same species. Two or more individuals are said to be allogenic to another when the genes at one or more loci are not identical.

As used herein, the term “autologous” as used herein shall be taken to mean from the same individual.

As used herein, the term “serum” refers to any type of serum derived from animals, preferably mammals, and more preferably from humans. In the context of the present application, the sera may be allogenic or autologous sera. The sera may be derived from a single individual or there may be pooled sera, for example mixed and pooled sera obtained from at least two individuals.

As used herein, the term “kit” refers to an arrangement of parts comprising, inter alia, a container, cells in a formulation, optionally further cell culture ingredients or any agreed ingredients, such as media or liquids for injection into a subject, a device for injection such as a syringe, injection pen, a needle, and optionally instructions for use, a holding device for the container, a cooling means, and the like.

As used herein, the term “adipose tissue” is meant any fat tissue. The adipose tissue may be brown or white adipose tissue, derived from subcutaneous, omental/visceral, mammary, gonadal or other tissue site. Typically, the adipose tissue is subcutaneous white adipose tissue. Such cells may comprise a primary cell culture or immortalized cell line. The adipose tissue may be from any organism having fat tissue. Typically, the adipose tissue is mammalian, most typically the adipose tissue is human. A convenient source of adipose tissue is from liposuction surgery but not limited to, however, the source of adipose tissue or the method of isolation of adipose tissue is not critical to the invention.

As used herein, an “individual to whom the cell product can be/is administered” may be selected from the group of human or animal patients or subjects, for example those suffering from cancer, immune diseases, patients subjected to chemotherapy, patients suffering from organ failure, from motor neuron disease, from acute and chronic organ dysfunction, from short- and long-term autoimmune disorders, neurodermatitis, osteoarthritis, myocardial dysfunction, systemic lupus erythematosus (SLE), psoriatic arthritis, fibromyalgia, stroke, diseases with organ degeneration, chronic obstructive lung disease, bone defects, inflammatory arthritis, spinal cord injury, limb wounds, liver fibrosis/cirrhosis, hepatobiliary diseases, neurodegenerative ophthalmic disorders, diabetes mellitus, inflammatory bowel diseases, graft-versus-host disease, polyneuropathy, sepsis, acute respiratory distress syndrome, non-arteritic ischemic optic neuropathy, nephropathy, although individuals may be those undergoing cosmetic treatment, for example those undergoing cosmetic treatment after hypertrophic burn scars, for breast reconstruction, et cetera.

As used herein, a “container which comprises a formulation comprising cells in the medium and a certain volume of gas” may comprise the formulation and the gas phase in form of two visually separate volumes, or the gas may be dissolved in the formulation.

As used herein, “viability” of the stored cells after a given storage time may be determined by means of any method known in the art, which are exemplified in the Examples section infra. However, other methods for the determination of the percentage of viable cells are equally suitable.

Herein below, various embodiments of the invention are explained in more detail. Wherever, respective alternatives in terms of ingredients in compositions, types of pharmaceutical compositions, concentrations of ingredients, periods of time of administration, frequencies of administration, medical indications to be treated are mentioned, the person skilled in the would immediately understand that individual combinations can be made as long as these are technically possible or if not otherwise explicitly indicated.

### DETAILED DESCRIPTION OF THE INVENTION

Irrespective of the cell type, a huge problem with living cells that have to be stored or shipped before they can be used, for example, in the treatment of patient, or before they can be further cultivated in vitro is the stability upon storage and the viability of the cells during storage and subsequently. These challenges are addressed by the present invention, which is explained below with respect to various embodiments. Whenever there is no technicality reasonable prejudice or fact not to combine any of the following embodiments, the present disclosure relates also to subject matter, in which two or more embodiments, or members of lists selected from groups may be read in combination.

### Embodiments

The product may be a packaged cell-containing product that does not require further cell culture steps before administration to an individual in need thereof. In that sense, a package cell product should not be confounded with a given container comprising cells in a culture medium that is not intended and/or suited for direct administration to an individual in need thereof.

In general, the cell-containing product takes the form of a formulation for direct administration. Of course, any residual gas phase present in the container comprising the formulation for direct administration should be removed before, e.g. injection into a patient. Only as an illustrative example, the packaged cell product of the present invention may be a syringe-like container or a vial for infusion comprising a formulation comprising cells to be administered to the individual in need thereof. The container may be fixed to an injection device, e.g. an injection needle, the residual gas phase may be removed through the injection needle before injection of the formulation comprising the cells to be injected, and subsequently said formulation is administered to an individual in need thereof.

The gas phase in the packaged cell product may be physically (and often visually) separated from the liquid phase comprising the formulation comprising the cells to be injected, or the gas phase may be dissolved in the liquid phase of the formulation comprised in the container. In either case, the formulation is suitable for direct administration to a person in need thereof, optionally after a step of removing residual gas.

The packaged cell product comprising the “ready-to-us” formulation comprising the respective cells is surprisingly storage stable and may be shipped at temperatures corresponding to chilled conditions rather than ultra-deep frozen conditions thereby avoiding thawing steps that are often associated with a substantial loss in the number of viable cells. However, it is also not excluded to ultra-deep freeze the packaged cell products according to the present invention. In preferred embodiments, however, no such ultra-deep freezing step is performed.

Subject matter of the present invention is a packaged cell product comprising a container comprising a formulation suitable for direct administration or direct injection to an individual in need thereof comprising cells in a medium, wherein the container comprises a gas phase in contact with said medium comprising between 1 to 100 Vol.-% of oxygen, particularly between 1 to 50 Vol.-% of oxygen, more particularly between 5 and 25 Vol.-% of oxygen.

Embodiments of the packaged cell products referred to above are those products that are kept under chilled conditions, e.g. at a temperature of 1-10° C., 2-8° C., at 1° C., 2° C., 3° C., 4° C., 5° C., 6° C., 7° C., 8° C., 9° C., and/or 10° C. The packaged cell product may comprise cells in a formulation that is inert and may be administered, e.g., injected into a person in need thereof. It is possible to up-concentrate the cells to be administered in a smaller volume of a formulation so that a small amount is injected. Ways of reducing the amount of formulation are, e.g., subjecting the container to a careful centrifugation step so as to not to harm the cells and removing part of the formulation that is present in the container. The formulation does generally not require a prior cultivation step, i.e. increasing the number of cells before administration under appropriate in vitro cultivation conditions is not required. The formulation in the packaged cell product may be warmed-up cautiously in order to administer or inject the cellular formulation at a temperature that corresponds approximately to the body temperature of the individual receiving the cellular formulation. In some embodiments the total volume of the administered cell formulation ranges from 1 to 100 mL but is not limited thereto. The formulation is generally physiologically acceptable and does not contain any particulate acellular matter such as carriers of cells (microbeads, etc.).

Subject matter of the invention is also the above-mentioned packaged cell product according, wherein said formulation is a therapeutic or prophylactic formulation or a formulation for cosmetic or tissue-restorative purposes.

Subject matter of the invention is also the above-mentioned packaged cell product, wherein said cells are selected from the group comprising stem cells, mesenchymal stem cells (MSCs) derived from a tissue selected from the group comprising fat tissue, bone marrow, umbilical cord, liver, dental pulp, synovial-fluid, retina, skeletal-muscle, periodontal ligament, urine, mammary gland, peripheral blood, Wharton's Jelly, periapical cyst, amnion, and amniotic fluid, embryonic stem cells, induced pluripotent stem cells, fetal stem cells, precursor cells, primary cells, somatic cells, T cells, Natural Killer T Cells, genetically modified cells comprising CART-cells, fibroblast type cells, pre-differentiated and differentiated cells.

Subject matter of the invention is also the above-mentioned packaged cell product, wherein said cells are selected from the group comprising mammalian cells, particularly human cells, more particularly human stem cells, human T cells and human cell lines.

Subject matter of the invention is also the above-mentioned packaged cell product, wherein said cells are selected from the group comprising cultured human stem cells, cultured human T cells, and cultured human cell lines.

Subject matter of the invention is also the above-mentioned packaged cell product, wherein said formulation comprises pooled human serum.

Subject matter of the invention is also the above-mentioned packaged cell product, wherein said gas phase comprising oxygen is selected from the group comprising ambient air, mixtures comprising oxygen and nitrogen, mixtures comprising oxygen and an inert gas selected from the group comprising Argon, Helium, Neon, Krypton and Xenon, pure oxygen, mixtures comprising CO₂.

Subject matter of the invention is also the above-mentioned packaged cell product, wherein said container is selected from the group comprising syringes, vials, phioles, vessels, cell culture flasks, tubes and transfusion bags.

Subject matter of the invention is also a kit comprising the packaged cell product and a device for injection into an individual in need thereof, optionally further comprising an injection needle and/or instructions for use.

Subject matter of the invention is also the above-mentioned kit, wherein said individual is selected from the group of patients suffering from cancer, immune diseases, patients subjected to chemotherapy, individuals undergoing cosmetic treatment, patients suffering from organ failure, from motor neuron disease, from acute and chronic organ dysfunction, from short- and long-term autoimmune disorders, from neurodermatitis, from osteoarthritis, from myocardial dysfunction, from systemic lupus erythematosus, from psoriatic arthritis, from fibromyalgia, from stroke, from diseases with organ degeneration, from chronic obstructive lung disease, from bone defects, from inflammatory arthritis, from spinal cord injury, from limb wounds, from liver fibrosis/cirrhosis, from hepatobiliary diseases, from neurodegenerative ophthalmic disorders, from diabetes mellitus, from inflammatory bowel diseases, from graft-versus-host disease, from polyneuropathy, from sepsis, from acute respiratory distress syndrome, from non-arteritic ischemic optic neuropathy, from nephropathy, and from individuals undergoing cosmetic treatment after hypertrophic burn scars and for breast reconstruction.

Subject matter of the invention is also the above-mentioned formulation for cell storage as defined above comprising human pooled serum, cells as defined above, and at least one dissolved gas as defined above, wherein said formulation is suitable for direct administration to an individual in need thereof.

Subject matter of the invention is also the above-mentioned formulation for cell storage, wherein said formulation is suitable for direct administration to an individual in need thereof, for use in the treatment and/or prevention of a disease, disorder or condition selected from the group comprising cancer, immune diseases, chemotherapeutically treated individuals, organ failure, motor neuron disease, acute and chronic organ dysfunction, short- and long-term autoimmune disorders, osteoarthritis, myocardial dysfunction, systemic lupus erythematosus, psoriatic arthritis, fibromyalgia, stroke, diseases with organ degeneration, chronic obstructive lung disease, bone defects, inflammatory arthritis, spinal cord injury, limb wounds, liver fibrosis/cirrhosis, hepatobiliary diseases, neurodegenerative ophthalmic disorders, diabetes mellitus, inflammatory bowel diseases, graft-versus-host disease, polyneuropathy, from sepsis, acute respiratory distress syndrome, non-arteritic ischemic optic neuropathy, nephropathy, and individuals undergoing cosmetic treatment after hypertrophic burn scars and for breast reconstruction.

Subject matter of the invention is also the above-mentioned formulation for cell storage as defined in above, wherein said formulation is suitable for direct administration to an individual in need thereof, for use in the treatment and/or prevention of a disease, disorder or condition selected from the group comprising cancer, immune diseases, chemotherapeutically treated individuals, organ failure, motor neuron disease, acute and chronic organ dysfunction, short- and long-term autoimmune disorders, neurodermatitis, osteoarthritis, myocardial dysfunction, systemic lupus erythematosus, psoriatic arthritis, fibromyalgia, stroke, diseases with organ degeneration, chronic obstructive lung disease, bone defects, inflammatory arthritis, spinal cord injury, limb wounds, liver fibrosis/cirrhosis, hepatobiliary diseases, neurodegenerative ophthalmic disorders, diabetes mellitus, inflammatory bowel diseases, graft-versus-host disease, polyneuropathy, from sepsis, acute respiratory distress syndrome, non-arteritic ischemic optic neuropathy, nephropathy, and individuals undergoing cosmetic treatment after hypertrophic burn scars and for breast reconstruction as defined above, and wherein said at least 50%, 60%, 70%, 80% 90% or more of said cells remain viable after storage in said formulation upon storage under chilled conditions, particularly chilled conditions between 2 to 8° C., for a period of at least 2 to 14 days, particularly for 2 to 10 days before use.

Subject matter of the invention is also the above-mentioned pharmaceutical composition comprising a formulation as defined above and uses thereof as therapeutic, prophylactic, restorative, alleviating composition for respective purposes.

Subject matter of the invention is also a method of treatment of an individual need thereof suffering from cancer, immune diseases, individuals subjected to chemotherapy, individuals undergoing cosmetic treatment, individuals suffering from organ failure, from motor neuron disease, from acute and chronic organ dysfunction, from short- and long-term autoimmune disorders, from neurodermatitis, from osteoarthritis, from myocardial dysfunction, from systemic lupus erythematosus, from psoriatic arthritis, from fibromyalgia, from stroke, from diseases with organ degeneration, from chronic obstructive lung disease, from bone defects, from inflammatory arthritis, from spinal cord injury, from limb wounds, from liver fibrosis/cirrhosis, from hepatobiliary diseases, from neurodegenerative ophthalmic disorders, from diabetes mellitus, from inflammatory bowel diseases, from graft-versus-host disease, from polyneuropathy, from sepsis, from acute respiratory distress syndrome, from non-arteritic ischemic optic neuropathy, from nephropathy, and individuals undergoing cosmetic treatment, particularly after hypertrophic burn scars and tissue reconstruction, particularly for breast reconstruction, comprising administering a formulation as defined above.

Subject matter of the present application is a packaged cell product comprising a container comprising a formulation comprising cells, wherein the container comprises at least 1% to 99%, 5% to 95%, 10% to 90%, 20% to 80%, 30% to 70%, and preferably 40% to 60%, for example, 50% of a gas or gas mixture as defined herein above. In embodiments of the present invention, the ratio between the formulation comprising cells and the gas phase is at least 1% to 99%, 5% to 95%, 10% to 90%, 20% to 80%, 30% to 70%, 40% to 60%, 50% to 50%, 60% to 40%, 70% to 30%, 75% to 25%, 80% to 20%, 90% to 10%, 95% to 5%, et cetera.

Subject matter of the present application is also a packaged cell product comprising a container that comprises a formulation which comprises the above-mentioned cells in a medium and a gas phase or gas mixture phase as defined in the preceding embodiment. The medium may be any cell storage medium that is suitable for the maintenance of the viability of at least 50%, preferably 60%, 70%, 80% 90% or more of the stored cells for at least 24 hours, preferably for at least 48 hours, more preferably for at least 72 hours, even more preferably for at least 96 hours, still more preferably for at least 120 hours, or for about 6, 7, 8, 9, 10 days or longer, in the inventive packaged cell product comprising a container. According to the present invention, the medium may be an aqueous-based medium or a medium that is not based on water (for example, isotonic salt solutions, water comprising 0.89 NaCl, media comprising fluorocarbons such as perfluorocarbons, which may be exemplified by LiquiVent (Allicance)) et cetera.

In accordance with the present invention, the packaged cell product comprising a container with a formulation that comprises the cells of interest permits the maintenance of the viability of at least 80% of the cells for at least 24 hours after storage.

In accordance with the present invention, the packaged cell product comprising a container with a formulation that comprises the cells of interest permits the maintenance of the viability of at least 80% of the cells for at least 48 hours after storage.

In accordance with the present invention, the packaged cell product comprising a container with a formulation that comprises the cells of interest permits the maintenance of the viability of at least 80% of the cells for at least 72 hours after storage.

In accordance with the present invention, the packaged cell product comprising a container with a formulation that comprises the cells of interest permits the maintenance of the viability of at least 80% of the cells for at least 96 hours after storage.

In accordance with the present invention, the packaged cell product comprising a container with a formulation that comprises the cells of interest permits the maintenance of the viability of at least 80% of the cells for at least 120 hours after storage.

In accordance with the present invention, the packaged cell product comprising a container with a formulation that comprises the cells of interest permits the maintenance of the viability of at least 90% of the cells for at least 24 hours after storage.

In accordance with the present invention, the packaged cell product comprising a container with a formulation that comprises the cells of interest permits the maintenance of the viability of at least 90% of the cells for at least 48 hours after storage.

In accordance with the present invention, the packaged cell product comprising a container with a formulation that comprises the cells of interest permits the maintenance of the viability of at least 90% of the cells for at least 72 hours after storage.

According to the present invention, the packaged cell product comprising a container with a formulation that comprises the cells of interest permits the maintenance of the viability of at least 90% of the cells for at least 96 hours after storage.

According to the present invention, the packaged cell product comprising a container with a formulation that comprises the cells of interest permits the maintenance of the viability of at least 90% of the cells for at least 120 hours after storage.

In accordance with the present invention, the packaged cell product comprising a container with a formulation that comprises the cells of interest permits the maintenance of the viability of at least 95% of the cells for at least 24 hours after storage.

In another aspect of the present invention, the packaged cell product comprising a container with a formulation that comprises the cells of interest permits the maintenance of the viability of at least 95% of the cells for at least 48 hours after storage.

In a further aspect of the present invention, the packaged cell product comprising a container with a formulation that comprises the cells of interest permits the maintenance of the viability of at least 95% of the cells for at least 72 hours after storage.

In a still further aspect of the present invention, the packaged cell product comprising a container with a formulation that comprises the cells of interest permits the maintenance of the viability of at least 95% of the cells for at least 96 hours after storage.

According to the present invention, the packaged cell product comprising a container with a formulation that comprises the cells of interest permits the maintenance of the viability of at least 95% of the cells for at least 120 hours after storage.

In embodiments of any one of the foregoing embodiments or aspects, the cells are animal cells, preferably human cells.

In embodiments of any of the foregoing embodiments or aspects, the cells are stem cells. In other embodiments, the cells are T cells, or CART-cells.

In embodiments of any of the foregoing embodiments or aspects, the cells are stem cells, in particular stem cells that are MSC's obtained from adipose tissue. In an aspect of the invention, the cells are human cells.

In embodiments of any of the foregoing embodiments or aspects, the gas or gas mixture in the packaged cell product comprises or is ambient air.

In embodiments of any of the foregoing embodiments or aspects, the gas or gas mixture in the packaged cell product comprises oxygen in an amount of 1 to 100%, preferably between 5 and 75% oxygen, more preferably between 10 and 50% of oxygen, or preferably between 10 and 40% of oxygen.

In embodiments of any of the foregoing embodiments or aspects, the gas or gas mixture in the packaged cell product comprises solved oxygen in a concentration of about 1 to 51.1 mg/l, 1 to 20 mg/l, and 1 to 15 mg/l in the liquid phase. In one particular embodiment, the gas or gas mixture in the packaged cell product comprises solved oxygen in a concentration when filled under standard pressure (≈101325 Pa) and cooled to a temperature of 2-25° C.

In accordance with the present invention, the gas or gas mixture in the packaged cell product comprises a technically increased oxygen concentration in the liquid phase when filled in under high pressure (>101325 Pa) and cooled to temperature conditions lower 2° C. As used herein, the term “technically increased oxygen concentration” means, for example, that the oxygen is solved in the medium that is not water-based, for example liquids from the group of fluorocarbons, sometimes referred to as perfluorocarbons or PFCs (organofluorine compounds with the formula CxFy) or perfluorooctane also known as octadecafluorooctane. It can also mean that the pressure of the gas phase is increased so that the amount of dissolved oxygen increases. Additionally, the cell product according to the invention may comprise a non-visible gas phase subsequent to technically increasing the oxygen concentration in the liquid phase.

In an aspect of the present invention, the formulation for cell storage in the container as defined in any of the foregoing embodiments or aspects comprises at least one ingredient selected from the group comprising human pooled serum, glucose, NaCl, serum or sera derived from animals, e.g., fetal serum, pooled serum, and buffered formulations, for example, PBS, HEPES, DMEM, alpha-MEM, TES, MOPS, BES and the like, etc.

In further embodiments according to any of the foregoing embodiments or aspects, the packaged cell product is present in a container, which may be selected from the group comprising syringes, vials, phials, cell culture flasks, tubes, cell culture bags, and the like.

In further embodiments according to any of the foregoing embodiments or aspects, a kit is provided, which comprises the packaged cell product according to any of the above aspects of the present invention, optionally in combination with a device for injection into an individual in need thereof, injection needle, a mandrel, a sterilization medium, instructions for use, patches, et cetera.

According to the invention, it is possible before administration to the subject or individual in need thereof, to let out of the gaseous phase, e.g. by slightly moving, shaking or tapping the formulation in the container, and opening the container so that the gauges phase is let out, whereas the formulation remains in the container. Subsequently, it is possible to attach a needle device, for example, a syringe, a mandrel, or any other suitable device, optionally attached to a tube, so that the formulation may be infused into said individual.

According to another aspect of the invention, the formulation according to the present invention is for use in the treatment and/or prevention/prophylaxis of a disease or disorder or condition in a human or animal patient, wherein said disease or disorder is selected from the group comprising cancer, immune diseases, chemotherapeutically treated individuals, organ failure, motor neuron disease, acute and chronic organ dysfunction, short- and long-term autoimmune disorders, osteoarthritis, myocardial dysfunction, systemic lupus erythematosus, psoriatic arthritis, fibromyalgia, stroke, diseases with organ degeneration, chronic obstructive lung disease, bone defects, inflammatory arthritis, spinal cord injury, limb wounds, liver fibrosis/cirrhosis, hepatobiliary diseases, neurodegenerative ophthalmic disorders, diabetes mellitus, inflammatory bowel diseases, graft-versus-host disease, polyneuropathy, from sepsis, acute respiratory distress syndrome, non-arteritic ischemic optic neuropathy, nephropathy, and individuals undergoing cosmetic treatment after hypertrophic burn scars, for breast reconstruction or for other restorative purposes, e.g. for tissue reconstruction of parts that have been damaged, e.g. by accidents, by diseases (e.g., cancer), etc.

As used herein, the expression “animal patient”, refers particularly to mammals, e.g. horses, dogs, cats, pigs, cattle, rodents, et cetera.

According to another aspect of the invention, the formulation according to the present invention is for use in the treatment and/or prevention/prophylaxis of a disease or disorder or a condition in a human or animal patient, wherein disease or disorder or a condition in a human or animal patient is selected from the group comprising cancer, immune diseases, chemotherapeutically treated individuals, organ failure, motor neuron disease, acute and chronic organ dysfunction, short- and long-term autoimmune disorders, neurodermatitis, osteoarthritis, myocardial dysfunction, systemic lupus erythematosus, psoriatic arthritis, fibromyalgia, stroke, diseases with organ degeneration, chronic obstructive lung disease, bone defects, inflammatory arthritis, spinal cord injury, limb wounds, liver fibrosis/cirrhosis, hepatobiliary diseases, neurodegenerative ophthalmic disorders, diabetes mellitus, inflammatory bowel diseases, graft-versus-host disease, polyneuropathy, from sepsis, acute respiratory distress syndrome, non-arteritic ischemic optic neuropathy, nephropathy, and individuals undergoing cosmetic treatment after hypertrophic burn scars and for breast reconstruction, and wherein the cells in the formulation according to the present invention/cell product comprising a formulation according to the present invention are stem cells, particularly MSCs, particularly derived from adipose tissue.

According to another aspect of the invention, the formulation according to the present invention is for use in the treatment and/or prevention/prophylaxis of a disease or disorder or a condition in a human or animal patient, wherein disease or disorder or a condition in a human or animal patient is cancer or an immune disease, and wherein the cells in the formulation according to the present invention/cell product comprising a formulation according to the present invention are CAR-T-Cells or genetically modified MSCs, particularly derived from adipose tissue.

According to another aspect of the invention, the formulation according to the present invention is for use in the treatment and/or prevention/prophylaxis of a disease or disorder or a condition in a human or animal patient, wherein disease or disorder or a condition in a human or animal patient wherein the cells in the formulation according to the present invention/cell product comprising a formulation according to the present invention are adult stem cells, such as MSCs, particularly derived from adipose tissue, optionally genetically modified MSCs, and wherein the disease is graft vs host disease, or wherein the disease is liver or renal failure, or wherein the disease is organ failure, e.g., liver or renal failure, or wherein the disease is an autoimmune disease, e.g., rheumatoid arthritis, or wherein the disease is neurodermatitits, or wherein the disease is osteoarthritis, or wherein the disease is myocardial dysfunction, or wherein the disease is chronic obstructive lung disease, or wherein the diseases, disorders or conditions are bone defects (which may also be treated with bone marrow derived MSC's), or wherein the disease is an inflammatory arthritis, or wherein the disease or condition is/are limb wounds, or wherein the disease or condition liver cirrhosis, or wherein the disease is diabetes mellitus, or wherein the condition is breast reconstruction, for example due to an underlying disease requiring the amputation of a breast, for example, breast cancer.

According to another aspect of the invention, the formulation according to the present invention is for use in the treatment and/or prevention/prophylaxis of a disease or disorder or a condition in a human or animal patient, wherein disease or disorder or a condition in a human or animal patient is selected from the group comprising cancer, immune diseases, chemotherapeutically treated individuals, organ failure, motor neuron disease, acute and chronic organ dysfunction, short- and long-term autoimmune disorders, neurodermatitis, osteoarthritis, myocardial dysfunction, systemic lupus erythematosus, psoriatic arthritis, fibromyalgia, stroke, diseases with organ degeneration, chronic obstructive lung disease, bone defects, inflammatory arthritis, spinal cord injury, limb wounds, liver fibrosis/cirrhosis, hepatobiliary diseases, neurodegenerative ophthalmic disorders, diabetes mellitus, inflammatory bowel diseases, graft-versus-host disease, polyneuropathy, from sepsis, acute respiratory distress syndrome, non-arteritic ischemic optic neuropathy, nephropathy, and individuals undergoing cosmetic treatment after hypertrophic burn scars and for breast reconstruction, and wherein the cells in the formulation according to the present invention/cell product comprising a formulation according to the present invention are stem cells, particularly MSCs, particularly derived from adipose tissue, wherein the cells had been chilled and stored before administration to the patient/treatment of the patient, wherein said formulation was previously stored, e.g., under chilled conditions between 2 to 8° C., and/or for a period of at least 2 to 14 days, particularly for 2 to 10 days before use.

According to another aspect of the invention, the formulation according to the present invention is for use in the treatment and/or prevention/prophylaxis of a disease or disorder or a condition in a human or animal patient, wherein disease or disorder or a condition in a human or animal patient is cancer or an immune disease, and wherein the cells in the formulation according to the present invention/cell product comprising a formulation according to the present invention are CAR-T-Cells or genetically modified MSCs, particularly derived from adipose tissue, wherein the cells had been chilled and stored before administration to the patient/treatment of the patient, wherein said formulation was previously stored, e.g., under chilled conditions between 2 to 8° C., and/or for a period of at least 2 to 14 days, particularly for 2 to 10 days before use.

According to another aspect of the invention, the formulation according to the present invention is for use in the treatment and/or prevention/prophylaxis of a disease or disorder or a condition in a human or animal patient, wherein disease or disorder or a condition in a human or animal patient wherein the cells in the formulation according to the present invention/cell product comprising a formulation according to the present invention are adult stem cells, such as MSCs, particularly derived from adipose tissue, optionally genetically modified MSCs, and wherein the disease is graft vs host disease, or wherein the disease is liver or renal failure, or wherein the disease is organ failure, e.g., liver or renal failure, or wherein the disease is an autoimmune disease, e.g., rheumatoid arthritis, or wherein the disease is neurodermatitis, or wherein the disease is osteoarthritis, or wherein the disease is myocardial dysfunction, or wherein the disease is chronic obstructive lung disease, or wherein the diseases, disorders or conditions are bone defects (which may also be treated with bone marrow derived MSC's), or wherein the disease is an inflammatory arthritis, or wherein the disease or condition is/are limb wounds, or wherein the disease or condition liver cirrhosis, or wherein the disease is diabetes mellitus, or wherein the condition is breast reconstruction, for example due to an underlying disease requiring the amputation of a breast, for example, breast cancer, wherein the cells had been chilled and stored before administration to the patient/treatment of the patient, wherein said formulation was previously stored, e.g., under chilled conditions between 2 to 8° C., and/or for a period of at least 2 to 14 days, particularly for 2 to 10 days before use.

Another aspect of the invention, relates to methods of treatment/prevention/prophylaxis of diseases or disorders comprising administering the formulation according to the present invention to a human or animal patient in need thereof, wherein said disease or disorder is selected from the group comprising cancer, immune diseases, chemotherapeutically treated individuals, organ failure, motor neuron disease, acute and chronic organ dysfunction, short- and long-term autoimmune disorders, neurodermatitis, osteoarthritis, myocardial dysfunction, systemic lupus erythematosus, psoriatic arthritis, fibromyalgia, stroke, diseases with organ degeneration, chronic obstructive lung disease, bone defects, inflammatory arthritis, spinal cord injury, limb wounds, liver fibrosis/cirrhosis, hepatobiliary diseases, neurodegenerative ophthalmic disorders, diabetes mellitus, inflammatory bowel diseases, graft-versus-host disease, polyneuropathy, from sepsis, acute respiratory distress syndrome, non-arteritic ischemic optic neuropathy, nephropathy, and individuals undergoing cosmetic treatment after hypertrophic burn scars and for breast reconstruction.

Another aspect of the invention, relates to methods of treatment/prevention/prophylaxis of diseases or disorders comprising administering the formulation according to the present invention to a human or animal patient in need thereof, wherein said disease or disorder is selected from the group comprising cancer, immune diseases, chemotherapeutically treated individuals, organ failure, motor neuron disease, acute and chronic organ dysfunction, short- and long-term autoimmune disorders, neurodermatitis, osteoarthritis, myocardial dysfunction, systemic lupus erythematosus, psoriatic arthritis, fibromyalgia, stroke, diseases with organ degeneration, chronic obstructive lung disease, bone defects, inflammatory arthritis, spinal cord injury, limb wounds, liver fibrosis/cirrhosis, hepatobiliary diseases, neurodegenerative ophthalmic disorders, diabetes mellitus, inflammatory bowel diseases, graft-versus-host disease, polyneuropathy, from sepsis, acute respiratory distress syndrome, non-arteritic ischemic optic neuropathy, nephropathy, and individuals undergoing cosmetic treatment after hypertrophic burn scars and for breast reconstruction, wherein the formulation comprises stem cells, for example MSCs, such as MSCs derived from adipose tissue. These cells can be human cells.

The invention now will be described with respect to the following examples; however, the scope of the present invention is not intended to be limited thereby. The following examples are to show the experimental data to confirm the usability and functionality of the formulation of this invention.

### Further Aspects of the Invention

a) A packaged cell product comprising a container comprising a formulation comprising cells in a medium, wherein the container comprises a gas phase comprising between 1 to 100 Vol.-% of oxygen, particularly between 1 to 50 Vol.-% of oxygen, more particularly between 5 and 25 Vol.-% of oxygen.
b) The packaged cell product according to aspect a), wherein said cells are selected from the group comprising stem cells, mesenchymal stem cells (MSCs) derived from a tissue selected from the group comprising fat tissue, bone marrow, umbilical cord, liver, dental pulp, synovial-fluid, retina, skeletal-muscle, periodontal ligament, urine, mammary gland, peripheral blood, Wharton's Jelly, periapical cyst, amnion, and amniotic fluid, embryonic stem cells, induced pluripotent stem cells, fetal stem cells, precursor cells, primary cells, somatic cells, T cells, Natural Killer T Cells, genetically modified cells comprising CART-cells, fibroblast type cells, pre-differentiated and differentiated cells, immortalized cell lines that may be selected from the group comprising HEK cells or HELA cells.
c) The packaged cell product according to aspects a) or b), wherein said cells are selected from the group comprising mammalian cells, particularly human cells, more particularly human stem cells, human T cells and human cell lines.
d) The packaged cell product according to aspects a) to c), wherein said cells are selected from the group comprising cultured human stem cells, cultured human T cells, and cultured human cell lines.
e) The packaged cell product according to aspects a) to d), wherein said formulation comprises pooled human serum.
f) The packaged cell product according to aspects a) to e), wherein said gas phase comprising oxygen is selected from the group comprising ambient air, mixtures comprising oxygen and nitrogen, mixtures comprising oxygen and an inert gas selected from the group comprising Argon, Helium, Neon, Krypton and Xenon, pure oxygen, mixtures comprising CO₂.
g) The packaged cell product according to aspects a) to f), wherein said container comprises is selected from the group comprising syringes, vials, phioles, cell culture flasks, tubes and transfusion bags.
h) The packaged cell product according to aspects a) to f), wherein dissolved oxygen in a concentration of 10-14 mg/l or at a technically increased oxygen concentration (≥14 mg/l) in the liquid phase.
i) A kit comprising the packaged cell product according to any of the preceding aspects and a device for injection into an individual in need thereof, optionally further comprising an injection needle, instructions for use.
j) The kit according to aspect i), wherein said individual is selected from the group of patients suffering from cancer, immune diseases, patients subjected to chemotherapy, individuals undergoing cosmetic treatment, patients suffering from organ failure, from motor neuron disease, from acute and chronic organ dysfunction, from short- and long-term autoimmune disorders, from neurodermatitis, from osteoarthritis, from myocardial dysfunction, from systemic lupus erythematosus, from psoriatic arthritis, from fibromyalgia, from stroke, from diseases with organ degeneration, from chronic obstructive lung disease, from bone defects, from inflammatory arthritis, from spinal cord injury, from limb wounds, from liver fibrosis/cirrhosis, from hepatobiliary diseases, from neurodegenerative ophthalmic disorders, from diabetes mellitus, from inflammatory bowel diseases, from graft-versus-host disease, from polyneuropathy, from sepsis, from acute respiratory distress syndrome, from non-arteritic ischemic optic neuropathy, from nephropathy, and from individuals undergoing cosmetic treatment after hypertrophic burn scars and for breast reconstruction.
k) A formulation for cell storage as defined in any of the preceding aspects comprising human pooled serum, cells as defined in any of the previous aspects, and at least one dissolved gas as defined in any of the foregoing aspects.
l) A formulation for cell storage as defined in aspect k) for use in the treatment and/or prevention of a disease, disorder or condition selected from the group comprising cancer, immune diseases, chemotherapeutically treated individuals, organ failure, motor neuron disease, acute and chronic organ dysfunction, short- and long-term autoimmune disorders, osteoarthritis, myocardial dysfunction, systemic lupus erythematosus, psoriatic arthritis, fibromyalgia, stroke, diseases with organ degeneration, chronic obstructive lung disease, bone defects, inflammatory arthritis, spinal cord injury, limb wounds, liver fibrosis/cirrhosis, hepatobiliary diseases, neurodegenerative ophthalmic disorders, diabetes mellitus, inflammatory bowel diseases, graft-versus-host disease, polyneuropathy, from sepsis, acute respiratory distress syndrome, non-arteritic ischemic optic neuropathy, nephropathy, and individuals undergoing cosmetic treatment after hypertrophic burn scars and for breast reconstruction.
m) A formulation for cell storage as defined in any of the previous aspects for use in the treatment and/or prevention of a disease, disorder or condition selected from the group comprising cancer, immune diseases, chemotherapeutically treated individuals, organ failure, motor neuron disease, acute and chronic organ dysfunction, short- and long-term autoimmune disorders, neurodermatitis, osteoarthritis, myocardial dysfunction, systemic lupus erythematosus, psoriatic arthritis, fibromyalgia, stroke, diseases with organ degeneration, chronic obstructive lung disease, bone defects, inflammatory arthritis, spinal cord injury, limb wounds, liver fibrosis/cirrhosis, hepatobiliary diseases, neurodegenerative ophthalmic disorders, diabetes mellitus, inflammatory bowel diseases, graft-versus-host disease, polyneuropathy, from sepsis, acute respiratory distress syndrome, non-arteritic ischemic optic neuropathy, nephropathy, and individuals undergoing cosmetic treatment after hypertrophic burn scars and for breast reconstruction according to aspect a), and wherein said formulation is stored under chilled conditions, particularly chilled conditions between 2 to 8° C., for a period of at least 2 to 14 days, particularly for 2 to 10 days before use.

### Experiments

### Introduction to Herein Used General Methods and Materials

Herein tested formulations comprise human pooled serum at a final concentration of 10% (w/v). Pooled human serum (Pool-Humanserum (P-HS)) obtained from “Zentrum für Klinische Transfusionsmedizin”, Tübingen/Germany was used, but pooled sera from other sources (e.g., fetal calf serum) or may also be used. The herein used formulation comprises glucose at a final concentration of 0.5% (w/v). The glucose was obtained as a solution from B. Braun (PZN:1307172), but other glucose solutions or formulations (e.g., in form of a powder) from other sources may equally be used. Dilutions performed in context of these experiments were made with isotonic sodium chloride solutions provided by B. Braun (PZN: 04272994), but other isotonic sodium chloride formulations (powder/solution) or buffered solutions from other sources may also be used. In the examples the liquid formulation was temperate to 2-25° C.

The herein tested formulations also comprise ambient air with an average oxygen amount of 21%. Formulations comprising a gas phase with an oxygen amount of ≥1% may also be used. The ratio for the gas and the fluid phase in the herein tested formulations is 1(g):2(f). Other ratios with an appropriate replacement of the oxygen in the fluid phase may also be used as shown, e.g., in Example 3. Solutions with dissolved oxygen at a concentration of 1-14 mg/l or a technical increased oxygen concentration (≥14 mg/l) may also be used. In the examples the containers were filled under standard pressure (≈101325 Pa).

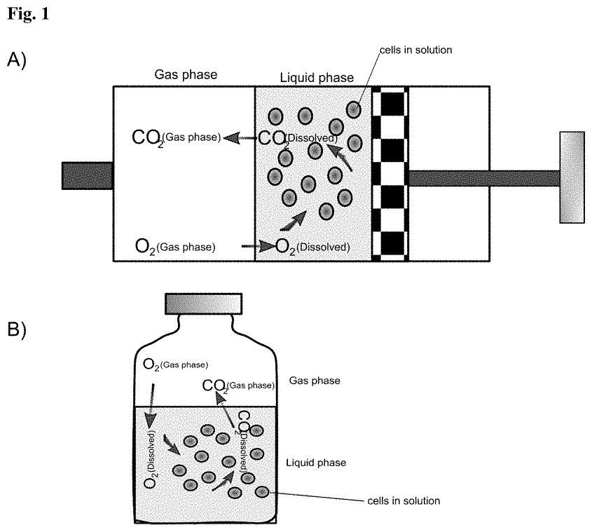

The herein tested formulations can be filled in syringes, e.g., those provided by B. Braun (2 mL PZN: 2057895; 5 mL PZN: 2057903; 10 mL: PZN: 2057926; 20 mL: PZN: 2057932) as illustrated in FIG. 1 A, but is not limited to them. It is possible to use different types of containers, e.g., those described below in Example 3 and FIG. 1 B.

In the exemplary formulations, the cell products comprise human mesenchymal stem cells derived from adipose tissue, but it would be possible also to use mesenchymal cells from other tissues, for example, those obtained by a bone marrow aspirate. As described in the above detailed specification, the stem cells may also be embryonic, fetal or induced. The origin of the cells may also be animal based. The mesenchymal stem cell may be autologous to the recipient, or may be allogenic to the recipient. The mesenchymal stem cells may be obtained by means known to those skilled in the art. For example, the mesenchymal stem cells may be obtained from adipose tissue or adipose aspirate, and the expanded in culture. Once expanded in culture, the mesenchymal stem cells can be stored in the formulation according to the present invention for 7 days under chilled conditions. Depending on cell type, longer storage periods are also possible. In general, the mesenchymal stem cells are dissolved at a concentration range of 1×10⁴-1×10⁹ cells/ml, preferably at a range from about 1×10⁵-1×10⁸ cells/ml, more preferably from about 1×10⁶-1×10⁷ cells/ml. The exact number of cells per volume depends on a variety of factors, including, but not limited to, the type and/or source of cells and the duration of the expansion period. An illustration of the herein described invention is provided in FIG. 1.

### EXAMPLE 1

The purpose of this experiment was to demonstrate the storage ability of MSC derived from adipose tissue (Adipose-derived stem cells/ASCs) as an example for MSCs in a formulation containing 10% (w/v) pooled human serum and 0.5% (w/v) glucose, diluted with isotonic sodium chloride solution. The formulations also contain ambient air (gas phase) and a fluid phase at a volume ratio of 1:2. The cells were stored under chilled conditions (2-8° C.) for several days (0-8 days).

### Method

The isolation protocol for human Adipose-derived stem cells out of the stromal vascular fraction was based on protocols described by Zuk et al. 2001 [3] and Zhu et al. 2013 [9] with modifications to achieve GMP-compliance. Briefly, lipoaspirate was washed and digested with Collagenase NB 6 GMP Grade (Nordmark) according to the manufacturer's recommendations for ˜35 minutes at 37° C. The digestion was stopped as described in the manual. After a centrifugation for 10 minutes (400 g, room temperature) the supernatant was discarded. For erythrocyte depletion the cells were further separated by Ficoll centrifugation (400 g, room temperature, 30 min./GE Healthcare Bio-Sciences) and seeded in cell culture flask. On the next day, the cells were washed with PBS (Phosphate Buffered Saline w/o Ca²⁺ or Mg²⁺) and expanded with culture media till confluency was reached (10-14 day corresponding to passage 0).

Expanded human ASCs (passages 0-11) were used for experimental purpose. To this end, the ASCs were harvested with trypsin and washed serval times with PBS (w/o Ca2⁺ or Mg²⁺), suspended in the above described formulation, filled in different syringes (2-20 ml Volume) under standard pressure (≈101325 Pa) conditions, packed in sterile bags and stored under chilled conditions for several day (0-8 days). After storage the cells were unpacked and the viability as well as the cell concentration was measured with Guava ViaCount Reagent (Merck-Millipore) according to the manufacturer's recommendations. The lowest cell-viability for a clinically usable cell product for humans or animals is ≥70%. The data were obtained by cells derived from 5 female donors with an average age of 28.2 years (range: 20-40 years). The phenotype of the herein used ASCs was verified by surface marker determination. All used ASCs were positively tested for the surface markers CD73, CD90 and CD105 and negatively tested for the surface marker CD34, CD45 and HLA-II as recommended in the literature [4].

### Results and Discussion

The mean viability for the mesenchymal stem cells after harvesting and washing was 94.5% (±1.7/n=39). Subsequently, the cells were diluted in the above described formulation with a mean cell concentration of 8.5×10⁶ cell/ml (±1.6×10⁶) and stored for serval days at 2-8° C. Results of the viability measurements represented as mean values are summarized in Table 1.

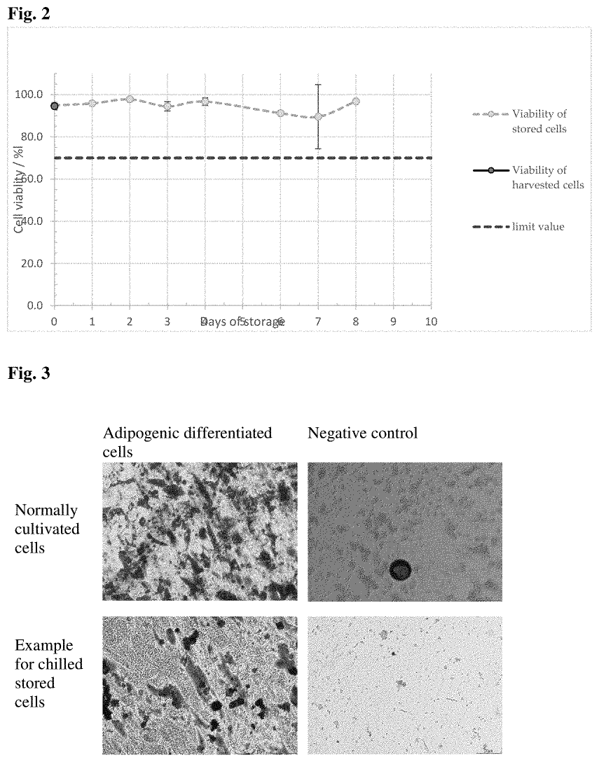

Further, in FIG. 2 the cell viability trend of the stored cells is shown. The data shown in Table 1 and FIG. 2 as compared with the cell viability after harvesting and diluting in the storage formulation (which corresponds to storage day zero) indicate that the herein used formulation has no harmful effect on the viability of the cells after diluting them. For days 1, 2, 3, 4, 6 and 8, stable cell viability over 90% could be shown. On Day 7, only 1 out of 25 cell products showed a reduced shelf life with a cell viability of 17.5%. All other viabilities were over 80%. This means that the cell product with a reduced shelf life is an outlier. Cell products containing living cells should be produced perfectly to avoid any problems. In the present case, the mean value for 7 days of storage of 89.5% cell viability is still surprisingly high above the limit value of 70%. Considering the fact that MSC-based cell products are very sensitive, only one outlier of 25 produced formulations of the invention is an exceptionally good result, which underlines the superior storage stability and viability of cells diluted in said formulation.

TABLE 1
Summary of the stored and
analyzed mesenchymal stem cells

| Storage time/d | 0 | 1 | 2 | 3 | 4 | 6 | 7 | 8 |
|---|---|---|---|---|---|---|---|---|
| Mean viability/% | 95.0 | 95.8 | 97.8 | 94.5 | 96.7 | 91.2 | 89.5 | 96.8 |
| SD | 1.0 | 0.5 | 0.9 | 2.2 | 1.8 | N/A | 15.2 | N/A |
| n | 3 | 2 | 2 | 4 | 2 | 1 | 25 | 1 |

N/A = not applicable

### EXAMPLE 2

This experiment intends to demonstrate that MSC based cell products are applicable after storage for seven days under chilled conditions. To proof the stemness of the stored mesenchymal stem cells, they were differentiated into the adipogenic, chondrogenic and osteogenic lineages as described in literature [4]. To show that MSCs stored for seven days have still clinical potential, the culture media of stored and expanded stem cells were collected and the amounts of secreted factors were measured. Additionally, the aging process of the stored cells was investigated. To this end, the senescence of these cells was estimated. Stem cells with a normal, i.e. non-tumorigenic life-cycle, show an increased senescence with cell age.

### Method

Expanded human ASCs as described in Example 1, were harvested with trypsin at passage 5, washed several times with PBS (w/o Ca²⁺ or Mg²⁺), suspended in the above described formulation of the invention, filled in syringes (5 ml Volume), packed in sterile bags and stored under chilled conditions for seven days. Subsequent to storage, cells were unpacked and seeded again in cell culture flasks. The cells were expanded until they reached a density of 70-90% (corresponding to passage 9). The culture media supernatant was collected and processed as explained below.

The MSCs were harvested with trypsin, washed several times with PBS (w/o Ca²⁺ or Mg²⁺). The phenotype of the used ASCs was verified by surface marker determination. All ASCs were positively tested for the surface markers CD73, CD90 and CD105 and negatively tested for the surface markers CD34, CD45 and HLA-II as recommended [4]. Subsequently the remaining MSCs were subjected to differentiation and senescence experiments.

The differentiation experiments were performed as described in Zhu et al. 2013 [9]. Briefly, for adipogenic and osteogenic differentiation 2.0×10⁵ cells/well were seeded in 24-well-plates. For chondrogenic “micromass culture”, 1.0×10⁵ cells/well were seeded in 24-well-plates. For all differentiation experiments StemMACS media (ChondroDiff Media, AdipoDiff Media and OsteoDiff Media) from Miltenyi Biotec GmbH were used according to the manufacturer's recommendations. After 14 days of cultivation with corresponding StemMACS media the differentiated cells were fixed and stained. In order to verify that differentiation was successful, adipocytes were stained with Oil Red-O-staining, chondrocytes with Alcian-blue-staining and osteoblasts with Kossa-staining. Pictures of the differentiated cells and the controls were taken with an inverse microscope Ts2-FL, DS-Fi3+DS (Nikon).

The senescence experiments were performed with a Senescence Cells Histochemical Staining Kit of Sigma-Aldrich Chemie GmbH according to the manufacturer's recommendations. Briefly, 2×10⁴ cells/well were seeded in 24-well-plates and cultured for 24 h. Subsequently, the cells were fixed and the Senescence-associated beta-galactosidase activity was verified by 5-bromo-4-chloro-3-indolyl-β-D-galactopyranoside (X-gal) staining. Additionally, the nucleus was stained with Bisbenzimide (Hoechst 33342/Thermo Fisher Scientific). Pictures of the cells were taken with an inverse microscope Ts2-FL, DS-Fi3+DS (Nikon). The number of senescence-positive cells and of total cells were counted using ImageJ (National Institutes of Health). Thereafter, the percentages of positive cells were calculated.

For the measurement of cytokines and grow factors in the above described culture media supernatants, the media was filtered through a syringe filter (0.22 μm/Merck-Millipore) and stored at −20° C. until the measurements were performed. To demonstrate the therapeutic potential of chilled stored ASCs the amount of b-NGF (nerve growth factor beta), Eotaxin, HGF (hepatocyte growth factor), IL-6 (interleukin 6), IL-8 (interleukin 8), MCP-1 (monocyte chemoattractant protein 1) and TNF-α (tumor necrosis factor alpha) were determined in the supernatants using a Bio-Plex Pro Assay Kit (BIO-RAD) and a Bio-Plex 200 Systems (BIO-RAD) according to manufacture protocols and manuals. Thereafter, the amounts of the factors in pg/ml were normalized to a cell number of 1×10⁶ CD90+ cells to compare the results.

### Results and Discussion

To find out if the storage of the cells under chilled conditions has any negative impact on the stemness, life cycle, aging of the cells, and/or therapeutic potential, respectively, three different experiments were performed. To this end, MSCs derived from adipose tissue (ASCs) were stored for seven days at 2-8° C. and re-seeded subsequently. Once the cells reached confluency the different experiments were performed and compared to normal cultivated cells.

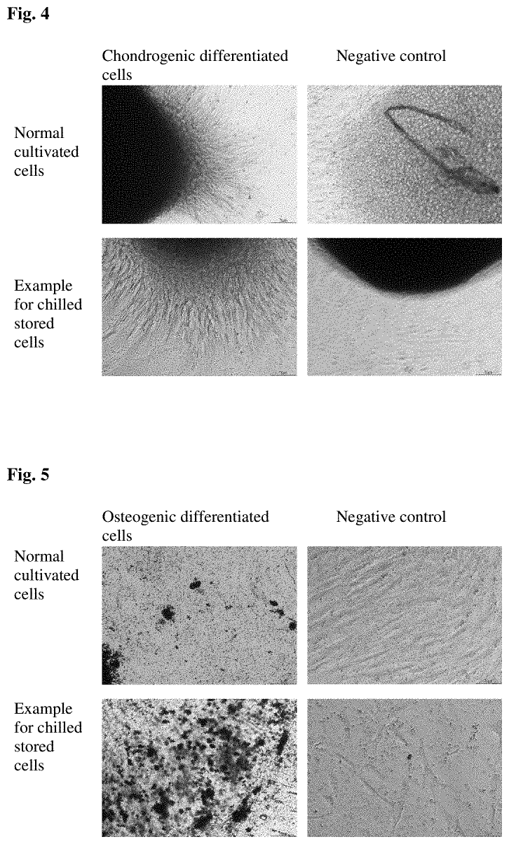

In the first experiment, the cells were differentiated in to adipocytes (FIG. 3), chondrocytes (FIG. 4) and osteoblasts (FIG. 5) to proof their stemness as described in literature [4]. As shown in FIGS. 3 to 5, chilled stored ASCs behave like normal cultivated cells when they were differentiated into the three lineages. No differences were seen which means that the cells kept their stemness upon storage.

The purpose of the second experiment was to demonstrate that storage under chilled conditions has no negative effect on the aging and life-cycle of the cells. To the end, the number of senescent cells was determined. Towards the end of their life cycle human cells express a (β-galactosidase. This enzyme can be detected with 5-bromo-4-chloro-3-indolyl-β-D-galactopyranoside (X-gal) staining [10].

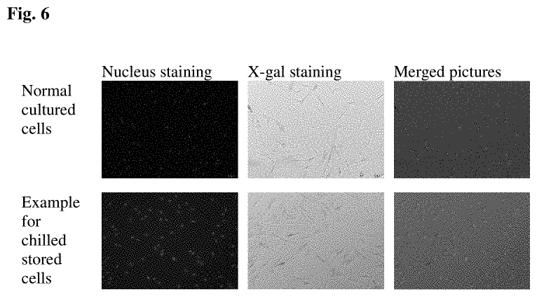

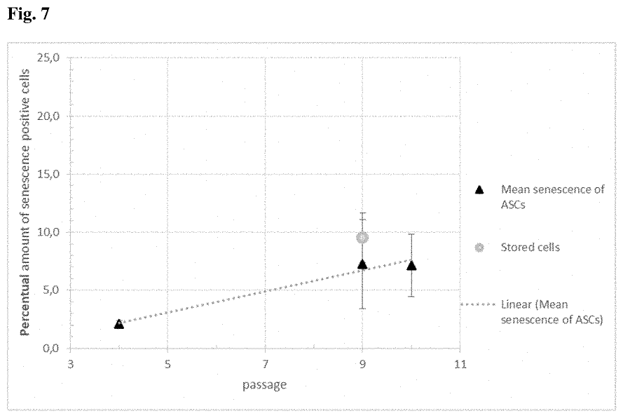

In FIG. 6 (middle column) the X-gal staining of normal cultivated cells is compared with the staining of chilled and stored MSCs. In these comparative examples, no differences could be detected. Further, the percentages of senescence-positive cells were calculated and compared (FIG. 7). To this end, the total number of the cells was counted on the basis of nucleus staining (FIG. 6/left column) As shown in FIG. 7 only a slight difference could be observed. This means that the storage under chilled conditions has no significant impact on the life-cycle. These tests also show that storage of MSCs at 2-8° C. in the inventive formulation does not lead to an increased abnormal cell aging.

One of the postulated modes of action in stem cell therapy described in literature [11-15] is the secretion of different mediators such as cytokines and grow factors.

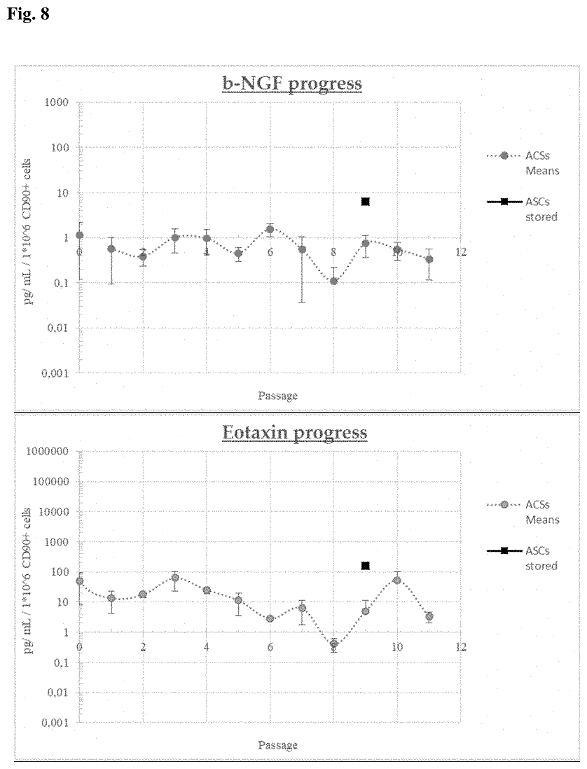

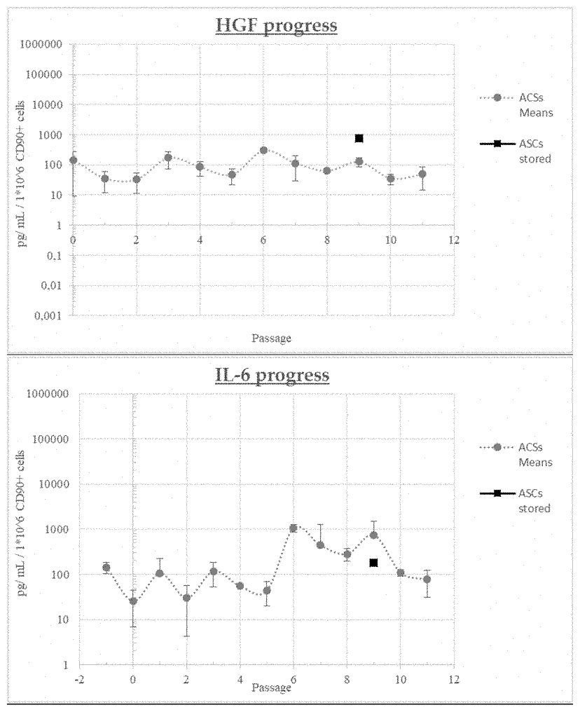

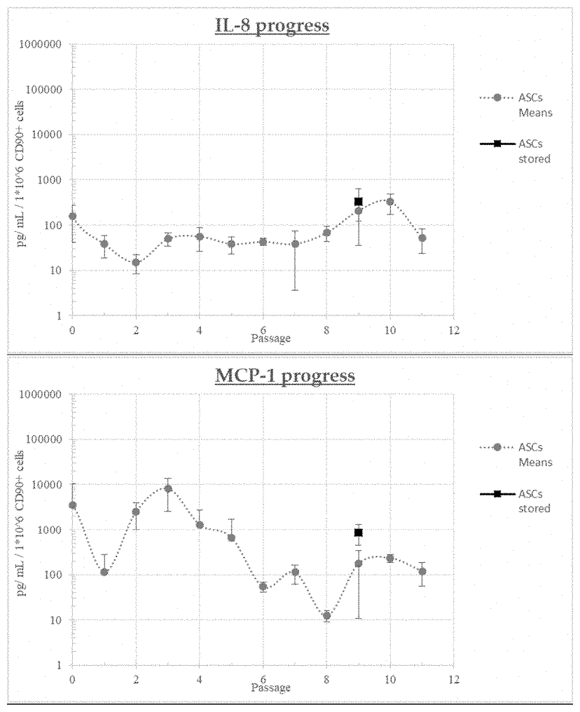

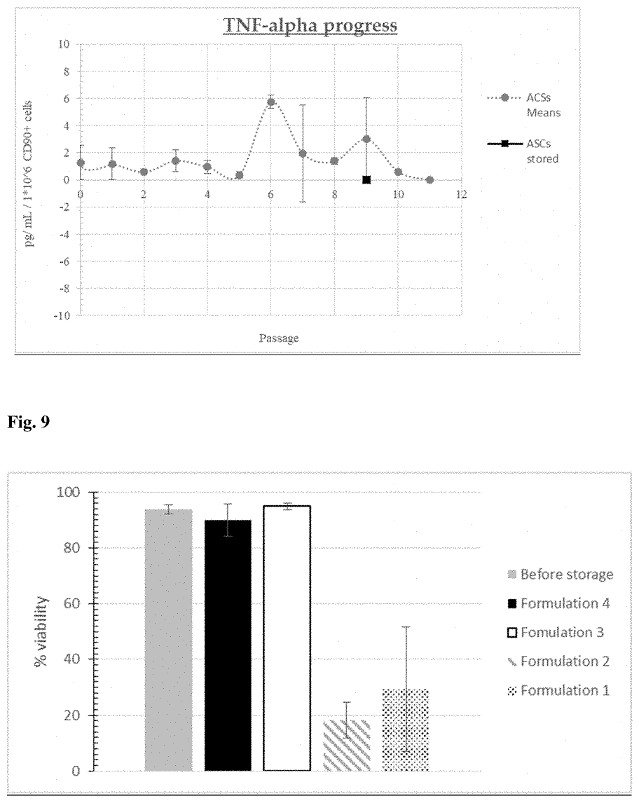

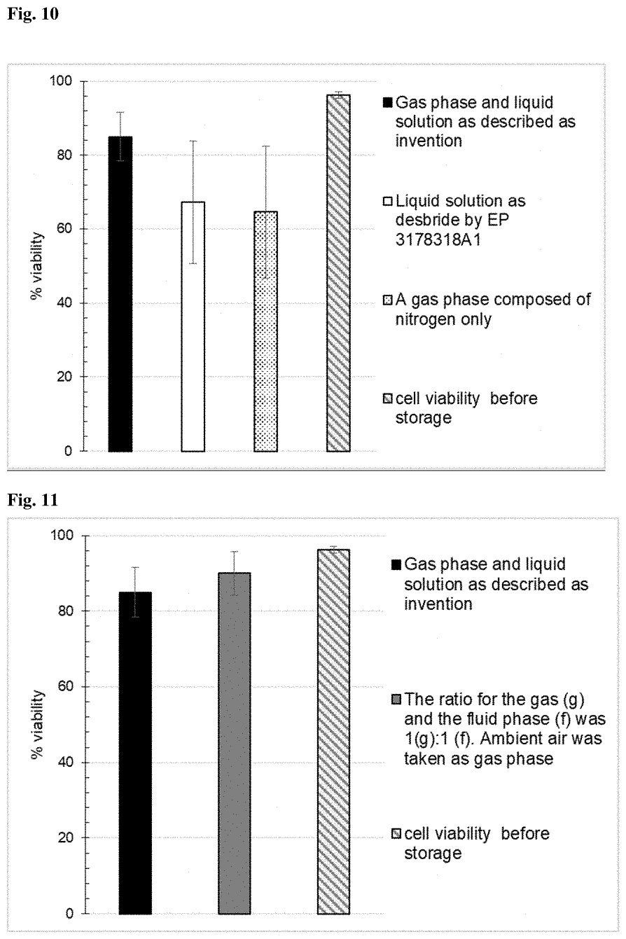

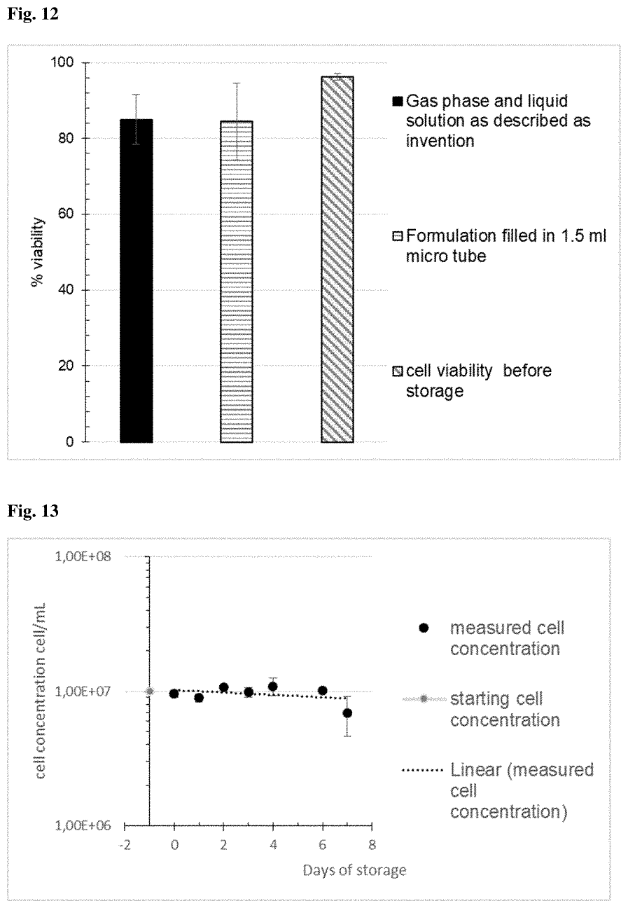

In the third experiment seven of the most common mediators [11-15] were chosen to compare the expression of these factors between normal cultivated cells and re-seeded cells stored previously for seven days under chilled conditions (FIG. 8).

For the mediators nerve growth factor beta (β-NGF), Eotaxin, hepatocyte growth factor (HGF) and monocyte chemoattractant protein 1 (MCP-1) a slight increase in expression was detected for cells stored previously under chilled conditions ((FIG. 8, black squares) compared to normal cultivated cells (FIG. 8, gray dots). For β-NGF, Eotaxin and HGF the expression seems to be higher than at any time-point measured for normal cultivated cells. In one further aspect of the invention it was shown, that the storage process has a positive benefit on the therapeutic usage of chilled stored stem cells. Therefore, the present invention relates also to the use of previously chilled and stored cells in the formulations described herein for use in the treatment and/or prevention of diseases, e.g., diseases selected from the group comprising cancer, immune diseases, patients subjected to chemotherapy, organ failure, motor neuron disease, acute and chronic organ dysfunction, short- and long-term autoimmune disorders, neurodermatitis, osteoarthritis, myocardial dysfunction, systemic lupus erythematosus, psoriatic arthritis, fibromyalgia, stroke, diseases with organ degeneration, chronic obstructive lung disease, bone defects, Inflammatory Arthritis, spinal cord injury, Limb Wounds, liver fibrosis/cirrhosis, hepatobiliary diseases, Neurodegenerative Ophthalmic Disorders, diabetes mellitus, inflammatory bowel diseases, graft-versus-host disease, polyneuropathy, sepsis, acute respiratory distress syndrome, non-arteritic ischemic optic neuropathy, and nephropathy, wherein said amelioration results from the administration of the immunomodulatory cells of the invention.

For the mediators, interleukin-6 (IL-6) and interleukin-8 (IL-8) no significant differences were detected when MSCs previously stored under chilled conditions were compared to normal cells. Tumor necrosis factor alpha (TNF-α) is measured for safety concerns because it belongs to the groups of endogenous pyrogens causing fever upon an infection with pathogens [16]. Therefore, the concentration of this factor should be as low as possible. For the cells according to the present invention which were stored under chilled conditions, the expression was slightly decreased compared to normal cultivated cells (FIG. 8). This clearly shows that cells stored at 2-8° C. for seven days are as safe as normal cultivated cells.

These experiments show clearly that the storage of cells in the inventive formulation under chilled conditions has no negative effect on the behavior of the cells. The cells behave as normal as cultivated ASCs that were not previously stored under chilled conditions. Surprisingly, a slight increase in the therapeutic potential was shown for MSCs which were stored under chilled conditions in accordance with the present invention underlining the advantage of using the same.

### Example 3

The purpose of this experiment was to investigate the role of the components in the formulation of the invention. The formulation of the cell products of the present invention consists of two phases, a liquid phase containing 10% (w/v) pooled human serum and 0.5% (w/v) glucose, diluted with isotonic sodium chloride solution. Further, the formulations comprise 1 volume gas phase (ambient air) and 2 volumes of fluid phase.

In a first part of the experiment, the influence of the components of the liquid phase was investigated. To this end, four different variations of the formulation were tested:
1. Sodium chloride alone,
2. Sodium chloride with glucose,
3. Sodium chloride with human pooled serum, and
4. Formulation described as invention.
   The gas phase was always ambient air.

The second aspect of the experiment paid attention to the composition of the gas phase described as invention and the used vessel to achieve the best mode. To this end, the same liquid formulation as above was used. Further, three different variations of the gas phase and another container than the usually used syringe were tested:
1. A variation of the ratio between gas phase and fluid phase,
2. A variation of the gas phase with another composition,
3. Only the liquid phase without a visible gas phase as described in EP 3178318 A1,
4. Another type of the container, i.e. not a syringe, and
5. A formulation as described as invention.

### Method

Expanded human ASCs as described in Example 1 were harvested with trypsin at passage 2-11, washed several times with PBS (w/o Ca²⁺ or Mg²⁺), suspended in four different formulations for experiment one, and five different formulations for experiment two as described above.

Briefly, for the first aspect of the experiment the formulations were as follows:
1. Isotonic sodium chloride solutions (B. Braun)
2. Glucose (B. Braun) diluted with isotonic sodium chloride solutions (B. Braun) to a final concentration of 0.5% (w/v)
3. Human pooled Serum (Zentrum für Klinische Transfusionsmedizin) diluted with isotonic sodium chloride solutions (B. Braun) to a final concentration of 10% (w/v)
4. Human pooled Serum (Zentrum für Klinische Transfusionsmedizin) diluted with isotonic sodium chloride solutions (B. Braun) to a final concentration of 10% (w/v) and Glucose (B. Braun) diluted with isotonic sodium chloride solutions (B. Braun) to a final concentration of 0.5% (w/v) as the described invention.

Subsequently, the solutions were mixed with the stem cells and filled in different syringes (≤1 ml Volume) with ambient gas. The volume ratio for the gas (g) and the fluid phase (f) was 1(g):2(f). Subsequently, the syringes were packed into sterile bags and stored under chilled conditions for seven days.

For the second aspect of this experiment the formulations of the liquid phase comprised human pooled Serum (Zentrum für Klinische Transfusionsmedizin) diluted with isotonic sodium chloride solutions (B. Braun) to a final concentration of 10% (w/v) and Glucose (B. Braun) diluted with isotonic sodium chloride solutions (B. Braun) to a final concentration of 0.5% (w/v) mixed with stem cells.

For the variations of the gas phase and the different type of container the following changes were performed:
1. The ratio for the gas and the fluid phase was 1(g):1(f). The gas phase consisted of ambient air.
2. The gas phase consisted of nitrogen (99.8% N₂ Gase Partner GmbH). The ratio of the gas (g) and the fluid phase (f) was 1(g):2(f).
3. The visible gas phase was excluded as much as possible, in accordance with the teaching in EP 3178318 A1.
4. The formulation as described in 5 below, but filled in a micro tube (1.5 ml/Sarstaedt) instead of a syringe.
5. Formulations according to the invention with a volume ratio of the gas (g) and the fluid (f) phase, respectively, that was 1(g):2(f).

The variations in number 1, 2, 3 and 5 above were filled into separate syringes (≤1 ml Volume), packed in sterile bags and stored under chilled conditions for seven days. Subsequent to the storage period the cells were unpacked and the viability as well as the cell concentration were measured with Guava ViaCount Reagent (Merck-Millipore) according to the manufacturer's recommendations. The phenotype of the used ASCs was verified by surface marker determination. All ASCs were positively tested for the surface markers CD73, CD90 and CD105 and negatively tested for the surface markers CD34, CD45 and HLA-II as recommended [4].

### Results and Discussion

In order to analyze the role of components on the liquid phase on the behavior of stem cells stored at 2-8° C., variants of the fluid phase were tested. In FIG. 9 the results of this experiment are shown. In the formulation according to Example 1 above (FIG. 9, black bar) and in a formulation containing 10% pooled human serum (FIG. 9, white bar) the viability of the chilled stored stem cells was similar to the start value FIG. 9, gray bar). This means that pooled human serum plays an important role in the formulation. The experiment also revealed that the glucose used in the formulation plays only a minor role (FIG. 9, gray diagonally lined bar).

In order to estimate the impact of the composition of the gas phase on the mesenchymal stem cells behavior, when these are stored job conditions, the second aspect of the experiment was performed as described above. Three different variations of the gas phase were compared to the formulation according to the invention. MSC's that were stored in a formulation with a visible gas phase of nitrogen, or in a formulation that did not include a gas phase visibly separated from the fluid phase as described in EP 3178318 A1 showed decreased stem cell viability compared to stem cells that were stored in the inventive formulations with a visible gas phase (FIG. 10). As shown herein the gas phase is important to increase the number of viable cells. Without oxygen the viability of the stem cells rapidly decreased. This clearly shows how important an oxygen containing visible gas phase is. (A few cells stored in a formulation with a visible gas phase of nitrogen or formulation without the gas phase remained viable, which may be due to the residual amount of oxygen dissolved in the respective solutions.)

As shown in FIG. 11, there is no difference between the MSCs stored in a formulation comprising the gas phase (g) to fluid phase (f) ratio of 2(g) to 1(f) or a gas phase (g) to fluid phase (f) ratio of 1(g) to 1(f). That means that it is possible according to the invention to use inventive formulations or sell products at different ratios of gas volume to liquid volume than used in example 1, preferably as long as a gas phase that is visibly separated from the fluid phase is present.

Furthermore, in order to demonstrate that the invention may also be carried out in other containers than syringes, a comparison between the formulations filled into syringes and a formulation filled into a microtube was conducted. As shown in FIG. 12 there is no significant difference between a syringe and a micro tube as a container. This means, that it is possible to use cell products in other containers than syringes. In summary, the above results of example 3 show that dissolved oxygen in a visible gas phase and pooled human serum having essential impact on the storage stability of stem cells when these are stored under chilled conditions defined herein. In addition, it could be shown that consuming oxygen should be replaced, which is made ashore by using a visible gas phase that comprises oxygen.

### Example 4

The purpose of this experiment was to point out the difference between a cell culture as well as cell expansion and a cell storage at conditions between 2-8° C. As described by Rieder et al. (2002) temperature lower 10° C. have an impact on the mammalian cell cycle [17]. Under this temperature cells stop dividing themselves and die. In comparison to cell culture and expansion at >36° C. There the cells dividing and growing. In consequent of this facts MSCs derived from adipose tissue (Adipose-derived stem cells/ASCs) as an example for MSCs solved in the solution described as invention and stored for several days (0-7 days) at 2-8° C. should show a stable or decreasing cell concentration.

### Method

Expanded human ASCs as described in Example 1 were harvested with trypsin at passage 2-11, washed several times with PBS (w/o Ca2+ or Mg2+), suspended in the formulation described as invention.

Subsequently, the solutions were mixed with the stem cells and filled in different syringes with ambient gas. The volume ratio for the gas (g) and the fluid phase (f) was 1(g):2(f). The starting concentration was 1.0×10⁷ cells/ml. Subsequently, the syringes were packed into sterile bags and stored under chilled conditions for several days.

After the storage period the cells were unpacked and the viability as well as the cell concentration were measured with Guava ViaCount Reagent (Merck-Millipore) according to the manufacturer's recommendations. The phenotype of the used ASCs was verified by surface marker determination. All ASCs were positively tested for the surface markers CD73, CD90 and CD105 and negatively tested for the surface markers CD34, CD45 and HLA-II as recommended [4].

### Results and Discussion

In order to show that during the cell storage no cell growth occurred, the cell concentration was measured subsequently. As shown in FIG. 13 the cell concentration which means the number of living cells, slightly decreased over the storage time. This clearly shows the differences to cell culture and expansion method described in EP 0 552 372 A1 or WO 2015/181185 A1. The aim of the methods described therein is to maximally increase the number of living cells. Additionally, experiments E1-E4 also clearly show the requirement of oxygen containing gas phases for the survival and quality of the stored live cells. This further distinguishes the herein described invention from the methods described in WO 2017/078654 A1 or by John et al. [18].

### REFERENCES

1. Maumus, M., D. Guerit, K. Toupet, et al., Mesenchymal stem cell-based therapies in regenerative medicine: applications in rheumatology. Stem Cell Res Ther, 2011. 2(2): p. 14.
2. Nauta, A. J. and W. E. Fibbe, Immunomodulatory properties of mesenchymal stromal cells. Blood, 2007. 110(10): p. 3499-506.
3. Zuk, P. A., M. Zhu, H. Mizuno, et al., Multilineage cells from human adipose tissue: implications for cell-based therapies. Tissue Eng, 2001. 7(2): p. 211-28.
4. Bourin, P., B. A. Bunnell, L. Casteilla, et al., Stromal cells from the adipose tissue-derived stromal vascular fraction and culture expanded adipose tissue-derived stromal/stem cells: a joint statement of the International Federation for Adipose Therapeutics and Science (IFATS) and the International Society for Cellular Therapy (ISCT). Cytotherapy, 2013. 15(6): p. 641-8.
5. Im, G. I., Y. W. Shin, and K. B. Lee, Do adipose tissue-derived mesenchymal stem cells have the same osteogenic and chondrogenic potential as bone marrow-derived cells? Osteoarthritis Cartilage, 2005. 13(10): p. 845-53.
6. Wagner, W., F. Wein, A. Seckinger, et al., Comparative characteristics of mesenchymal stem cells from human bone marrow, adipose tissue, and umbilical cord blood. Exp Hematol, 2005. 33(11): p. 1402-16.
7. Jorgensen, C., Final Report Summary—ADIPOA (ADIPOSE DERIVED STROMAL CELLS FOR OSTEOARTHRITIS). 2015, Community Research and Development Information Service
8. Galvez-Martin, P., A. Hmadcha, B. Soria, et al., Study of the stability of packaging and storage conditions of human mesenchymal stem cell for intra-arterial clinical application in patient with critical limb ischemia. Eur J Pharm Biopharm, 2014. 86(3): p. 459-68.
9. Zhu, M., S. Heydarkhan-Hagvall, M. Hedrick, et al., Manual isolation of adipose-derived stem cells from human lipoaspirates. J Vis Exp, 2013(79): p. e50585.
10. Dimri, G. P., X. Lee, G. Basile, et al., A biomarker that identifies senescent human cells in culture and in aging skin in vivo. Proceedings of the National Academy of Sciences of the United States of America, 1995. 92(20): p. 9363-9367.
11. Neuss, S., E. Becher, M. Woltje, et al., Functional expression of HGF and HGF receptor/c-met in adult human mesenchymal stem cells suggests a role in cell mobilization, tissue repair, and wound healing. Stem Cells, 2004. 22(3): p. 405-14.
12. Han, C., L. Zhang, L. Song, et al., Human adipose-derived mesenchymal stem cells: a better cell source for nervous system regeneration. Chin Med J (Engl), 2014. 127(2): p. 329-37.
13. Wang, F., N. Baba, Y. Shen, et al., CCL11 promotes migration and proliferation of mouse neural progenitor cells. Stem Cell Res Ther, 2017. 8(1): p. 26.
14. Banas, A., T. Teratani, Y. Yamamoto, et al., IFATS collection: in vivo therapeutic potential of human adipose tissue mesenchymal stem cells after transplantation into mice with liver injury. Stem Cells, 2008. 26(10): p. 2705-12.
15. Salcedo, R., H. A. Young, M. L. Ponce, et al., Eotaxin (CCL11) Induces In Vivo Angiogenic Responses by Human CCR3+ Endothelial Cells. The Journal of Immunology, 2001. 166(12): p. 7571-7578.
16. Locksley, R. M., N. Killeen, and M. J. Lenardo, The TNF and TNF receptor superfamilies: integrating mammalian biology. Cell, 2001. 104(4): p. 487-501.
17. Rieder, C. L. and R. W. Cole, Cold-Shock and the Mammalian Cell Cycle. Cell Cycle, 2002. 1(3): p. 168-174.
18. John, J., A. Dalgleish, A. Melcher, et al., Cryopreserved dendritic cells for intratumoral immunotherapy do not require re-culture prior to human vaccination. J Immunol Methods, 2005. 299(1-2): p. 37-46.

1. A packaged cell product comprising a container comprising a formulation suitable for direct administration or direct injection to an individual in need thereof comprising cells in a medium, wherein the container comprises a gas phase in contact with said medium comprising between 1 to 100 Vol.-% of oxygen, particularly between 1 to 50 Vol.-% of oxygen, more particularly between 5 and 25 Vol.-% of oxygen.

2. The packaged cell product according to claim 1, wherein said formulation is a therapeutic or prophylactic formulation or a formulation for cosmetic or tissue-restorative purposes.

3. The packaged cell product according to claim 1, wherein said cells are selected from the group comprising stem cells, mesenchymal stem cells (MSCs) derived from a tissue selected from the group comprising fat tissue, bone marrow, umbilical cord, liver, dental pulp, synovial-fluid, retina, skeletal-muscle, periodontal ligament, urine, mammary gland, peripheral blood, Wharton's Jelly, periapical cyst, amnion, and amniotic fluid, embryonic stem cells, induced pluripotent stem cells, fetal stem cells, precursor cells, primary cells, somatic cells, T cells, Natural Killer T Cells, genetically modified cells comprising CART-cells, fibroblast type cells, pre-differentiated and differentiated cells.

4. The packaged cell product according to claim 1, wherein said cells are selected from the group comprising mammalian cells, particularly human cells, more particularly human stem cells, human T cells and human cell lines.

5. The packaged cell product according to claim 1, wherein said cells are selected from the group comprising cultured human stem cells, cultured human T cells, and cultured human cell lines.

6. The packaged cell product according to claim 1, wherein said formulation comprises pooled human serum.

7. The packaged cell product according to claim 1, wherein said gas phase comprising oxygen is selected from the group comprising ambient air, mixtures comprising oxygen and nitrogen, mixtures comprising oxygen and an inert gas selected from the group comprising Argon, Helium, Neon, Krypton and Xenon, pure oxygen, mixtures comprising CO₂.

8. The packaged cell product according to claim 1, wherein said container is selected from the group comprising syringes, vials, phioles, cell culture flasks, tubes and transfusion bags.

9. A kit comprising the packaged cell product according to claim 1 and a device for injection into an individual in need thereof, optionally further comprising an injection needle and/or instructions for use.

10. The kit according to claim 9, wherein said individual is selected from the group of patients suffering from cancer, immune diseases, patients subjected to chemotherapy, individuals undergoing cosmetic treatment, patients suffering from organ failure, from motor neuron disease, from acute and chronic organ dysfunction, from short- and long-term autoimmune disorders, from neurodermatitis, from osteoarthritis, from myocardial dysfunction, from systemic lupus erythematosus, from psoriatic arthritis, from fibromyalgia, from stroke, from diseases with organ degeneration, from chronic obstructive lung disease, from bone defects, from inflammatory arthritis, from spinal cord injury, from limb wounds, from liver fibrosis/cirrhosis, from hepatobiliary diseases, from neurodegenerative ophthalmic disorders, from diabetes mellitus, from inflammatory bowel diseases, from graft-versus-host disease, from polyneuropathy, from sepsis, from acute respiratory distress syndrome, from non-arteritic ischemic optic neuropathy, from nephropathy, and from individuals undergoing cosmetic treatment after hypertrophic burn scars and for breast reconstruction.

11. A formulation for cell storage comprising human pooled serum, as defined in claim 1, and at least one dissolved gas, wherein said formulation is suitable for direct administration to an individual in need thereof.

12. A formulation for cell storage as defined in claim 11, wherein said formulation is suitable for direct administration to an individual in need thereof, for use in the treatment and/or prevention of a disease, disorder or condition selected from the group comprising cancer, immune diseases, chemotherapeutically treated individuals, organ failure, motor neuron disease, acute and chronic organ dysfunction, short- and long-term autoimmune disorders, osteoarthritis, myocardial dysfunction, systemic lupus erythematosus, psoriatic arthritis, fibromyalgia, stroke, diseases with organ degeneration, chronic obstructive lung disease, bone defects, inflammatory arthritis, spinal cord injury, limb wounds, liver fibrosis/cirrhosis, hepatobiliary diseases, neurodegenerative ophthalmic disorders, diabetes mellitus, inflammatory bowel diseases, graft-versus-host disease, polyneuropathy, from sepsis, acute respiratory distress syndrome, non-arteritic ischemic optic neuropathy, nephropathy, and individuals undergoing cosmetic treatment after hypertrophic burn scars and for breast reconstruction.

13. A formulation for cell storage as defined claim 11, wherein said formulation is suitable for direct administration to an individual in need thereof, for use in the treatment and/or prevention of a disease, disorder or condition selected from the group comprising cancer, immune diseases, chemotherapeutically treated individuals, organ failure, motor neuron disease, acute and chronic organ dysfunction, short- and long-term autoimmune disorders, neurodermatitis, osteoarthritis, myocardial dysfunction, systemic lupus erythematosus, psoriatic arthritis, fibromyalgia, stroke, diseases with organ degeneration, chronic obstructive lung disease, bone defects, inflammatory arthritis, spinal cord injury, limb wounds, liver fibrosis/cirrhosis, hepatobiliary diseases, neurodegenerative ophthalmic disorders, diabetes mellitus, inflammatory bowel diseases, graft-versus-host disease, polyneuropathy, from sepsis, acute respiratory distress syndrome, non-arteritic ischemic optic neuropathy, nephropathy, and individuals undergoing cosmetic treatment after hypertrophic burn scars and for breast reconstruction and
wherein said at least 50%, 60%, 70%, 80% 90% or more of said cells remain viable after storage in said formulation upon storage under chilled conditions, particularly chilled conditions between 2 to 8° C., for a period of at least 2 to 14 days, particularly for 2 to 10 days before use.

14. A pharmaceutical composition comprising a formulation as defined claim 11.

15. A method of treatment of an individual in need thereof suffering from cancer, immune diseases, individuals subjected to chemotherapy, individuals undergoing cosmetic treatment, individuals suffering from organ failure, from motor neuron disease, from acute and chronic organ dysfunction, from short- and long-term autoimmune disorders, from neurodermatitis, from osteoarthritis, from myocardial dysfunction, from systemic lupus erythematosus, from psoriatic arthritis, from fibromyalgia, from stroke, from diseases with organ degeneration, from chronic obstructive lung disease, from bone defects, from inflammatory arthritis, from spinal cord injury, from limb wounds, from liver fibrosis/cirrhosis, from hepatobiliary diseases, from neurodegenerative ophthalmic disorders, from diabetes mellitus, from inflammatory bowel diseases, from graft-versus-host disease, from polyneuropathy, from sepsis, from acute respiratory distress syndrome, from non-arteritic ischemic optic neuropathy, from nephropathy, and individuals undergoing cosmetic treatment, particularly after hypertrophic burn scars and tissue reconstruction, particularly for breast reconstruction, comprising administering a formulation as defined in claim 11.
